SEQUENCE LISTING (98 sequences)

SEQ ID NO: 1 - protein, 241 aa, Homo sapiens
  misc_feature: Human DNGR-1 (CLEC9A)
MHEEEIYTSLQWDSPAPDTYQKCLSSNKCSGACCLVMVISCVFCMGLLTASIFLGVKLLQVSTIAMQQQEKLIQQERALLNFTEWKRSCALQMKYCQAFMQNSLSSAHNSSPCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYALRSSV

SEQ ID NO: 2 - protein, 32 aa, Artificial Sequence
  The cytoplasmic domain of human DNGR-1
MHEEEIYTSLQWDSPAPDTYQKCLSSNKCSGA

SEQ ID NO: 3 - protein, 24 aa, Artificial Sequence
  The transmembrane domain of human DNGR-1
CCLVMVISCVFCMGLLTASIFLGV

SEQ ID NO: 4 - protein, 55 aa, Artificial Sequence
  The neck domain of human DNGR-1
KLLQVSTIAMQQQEKLIQQERALLNFTEWKRSCALQMKYCQAFMQNSLSSAHNSS

SEQ ID NO: 5 - protein, 125 aa, Artificial Sequence
  The C-type lectin domain (CTLD) of human DNGR-1
PCPNNWIQNRESCYYVSEIWSIWHTSQENCLKEGSTLLQIESKEEMDFITGSLRKIKGSYDYWVGLSQDGHSGRWLWQDGSSPSPGLLPAERSQSANQVCGYVKSNSLLSSNCSTWKYFICEKYA

SEQ ID NO: 6 - protein, 264 aa, Mus musculus
  misc_feature: Mouse DNGR-1 (CLEC9A)
MHAEEIYTSLQWDIPTSEASQKCQSPSKCSGAWCVVTMISCVVCMGLLATSIFLGIKFFQVSSLVLEQQERLIQQDTALVNLTQWQRKYTLEYCQALLQRSLHSGTDASTGPVLLTSPQMVPQTLDSKETGSDCSPCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKAFGSCI

SEQ ID NO: 7 - protein, 32 aa, Artificial Sequence
  The cytoplasmic domain of mouse DNGR-1
MHAEEIYTSLQWDIPTSEASQKCQSPSKCSGA

SEQ ID NO: 8 - protein, 24 aa, Artificial Sequence
  The transmembrane domain of mouse DNGR-1
WCVVTMISCVVCMGLLATSIFLGI

SEQ ID NO: 9 - protein, 79 aa, Artificial Sequence
  The neck domain of mouse DNGR-1
KFFQVSSLVLEQQERLIQQDTALVNLTQWQRKYTLEYCQALLQRSLHSGTDASTGPVLLTSPQMVPQTLDSKETGSDCS

SEQ ID NO: 10 - protein, 124 aa, Artificial Sequence
  The C-type lectin domain (CTLD) of mouse DNGR-1
PCPHNWIQNGKSCYYVFERWEMWNISKKSCLKEGASLFQIDSKEEMEFISSIGKLKGGNKYWVGVFQDGISGSWFWEDGSSPLSDLLPAERQRSAGQICGYLKDSTLISDKCDSWKYFICEKKA

SEQ ID NO: 11 - protein, 13 aa, Artificial Sequence
  The signalling domain of the cytoplasmic tail of DNGR-1
MHAEEIYTSLQWD

SEQ ID NO: 12 - protein, 7 aa, Artificial Sequence
  hemITAM domain
EEIYTSL

SEQ ID NO: 13 - protein, 8 aa, Artificial Sequence
  hemITAM domain
  misc_feature (6)..(7): Xaa can be any naturally occurring amino acid
DEDGYXXL

SEQ ID NO: 14 - protein, 7 aa, Artificial Sequence
  hemITAM domain
  misc_feature (2)..(3): Xaa can be any naturally occurring amino acid
  misc_feature (5)..(6): Xaa can be any naturally occurring amino acid
EXXYXXL

SEQ ID NO: 15 - protein, 13 aa, Artificial Sequence
  intracellular signalling domain
  misc_feature (5)..(6): Xaa can be any naturally occurring amino acid
  misc_feature (8)..(9): Xaa can be any naturally occurring amino acid
MHAEXXYXXLQWD

SEQ ID NO: 16 - protein, 244 aa, Mus musculus
  misc_feature: Mouse Dectin-1 (CLEC7A)
MKYHSHIENLDEDGYTQLDFSTQDIHKRPRGSEKGSRAPSSPWRPIAVGLGILCFVVVVVAAVLGALAFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICEKEL

SEQ ID NO: 17 - protein, 44 aa, Artificial Sequence
  The cytoplasmic domain of mouse Dectin-1
MKYHSHIENLDEDGYTQLDFSTQDIHKRPRGSEKGSRAPSSPWR

SEQ ID NO: 18 - protein, 23 aa, Artificial Sequence
  The transmembrane domain of mouse Dectin-1
VGLGILCFVVVVVAAVLGALAFW

SEQ ID NO: 19 - protein, 55 aa, Artificial Sequence
  The neck domain of mouse Dectin-1
RHNSGRNPEEKDSFLSRNKENHKPTESSLDEKVAPSKASQTTGGFSQSCLPNWIM

SEQ ID NO: 20 - protein, 116 aa, Artificial Sequence
  The C-type lectin domain (CTLD) of mouse Dectin-1
HGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICE

SEQ ID NO: 21 - protein, 86 aa, Homo sapiens
  misc_feature: Human FcR gamma chain
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 22 - protein, 42 aa, Artificial Sequence
  The cytoplasmic domain of human FcR gamma chain
RLKIQVRKAAITSYEKSDGVYTGLSTRNQETYETLKHEKPPQ

SEQ ID NO: 23 - protein, 21 aa, Artificial Sequence
  The transmembrane domain of human FcR gamma chain
LCYILDAILFLYGIVLTLLYC

SEQ ID NO: 24 - protein, 5 aa, Artificial Sequence
  The extracellular domain of human FcR gamma chain
LGEPQ

SEQ ID NO: 25 - protein, 86 aa, Mus musculus
  misc_feature: Mouse FcR gamma chain
MISAVILFLLLLVEQAAALGEPQLCYILDAVLFLYGIVLTLLYCRLKIQVRKAAIASREKADAVYTGLNTRSQETYETLKHEKPPQ

SEQ ID NO: 26 - protein, 42 aa, Artificial Sequence
  The cytoplasmic domain of mouse FcR gamma chain
RLKIQVRKAAIASREKADAVYTGLNTRSQETYETLKHEKPPQ

SEQ ID NO: 27 - protein, 21 aa, Artificial Sequence
  The transmembrane domain of mouse FcR gamma chain
LCYILDAVLFLYGIVLTLLYC

SEQ ID NO: 28 - protein, 5 aa, Artificial Sequence
  The extracellular domain of mouse FcR gamma chain
LGEPQ

SEQ ID NO: 29 - protein, 374 aa, Homo sapiens
  misc_feature: Human Fc gamma RI
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHVLFYLAVGIMFLVNTVLWVTIRKELKRKKKWDLEISLDSGHEKKVISSLQEDRHLEEELKCQEQKEEQLQEGVHRKEPQGAT

SEQ ID NO: 30 - protein, 61 aa, Artificial Sequence
  The cytoplasmic domain of human Fc gamma RI
RKELKRKKKWDLEISLDSGHEKKVISSLQEDRHLEEELKCQEQKEEQLQEGVHRKEPQGAT

SEQ ID NO: 31 - protein, 21 aa, Artificial Sequence
  The transmembrane domain of human Fc gamma RI
VLFYLAVGIMFLVNTVLWVTI

SEQ ID NO: 32 - protein, 277 aa, Artificial Sequence [sequence omitted]

SEQ ID NO: 33 - protein, 404 aa, Mus musculus
  misc_feature: Mouse Fc gamma RI
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVKIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 34 - protein, 84 aa, Artificial Sequence
  The cytoplasmic domain of mouse Fc gamma RI
KIHRLQREKKYNLEVPLVSEQGKKANSFQQVRSDGVYEEVTATASQTTPKEAPDGPRSSVGDCGPEQPEPLPPSDSTGAQTSQS

SEQ ID NO: 35 - protein, 23 aa, Artificial Sequence
  The transmembrane domain of mouse Fc gamma RI
VWFHILFYLSVGIMFSLNTVLYV

SEQ ID NO: 36 - protein, 273 aa, Artificial Sequence
  The extracellular domain of mouse Fc gamma RI
EVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAP

SEQ ID NO: 37 - protein, 317 aa, Homo sapiens
  misc_feature: Human Fc gamma RIIA
MTMETQMSQNVCPRNLWLLQPLTVLLLLASADSQAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 38 - protein, 77 aa, Artificial Sequence
  The cytoplasmic domain of human Fc gamma RIIA
CRKKRISANSTDPVKAAQFEPPGRQMIAIRKRQLEETNNDYETADGGYMTLNPRAPTDDDKNIYLTLPPNDHVNSNN

SEQ ID NO: 39 - protein, 23 aa, Artificial Sequence
  The transmembrane domain of human Fc gamma RIIA
IIVAVVIATAVAAIVAAVVALIY

SEQ ID NO: 40 - protein, 184 aa, Artificial Sequence
  The extracellular domain of human Fc gamma RIIA
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMG

SEQ ID NO: 41 - protein, 378 aa, Homo sapiens
  misc_feature: Human TIMD4
MSKEPLILWLMIEFWWLYLTPVTSETVVTEVLGHRVTLPCLYSSWSHNSNSMCWGKDQCPYSGCKEALIRTDGMRVTSRKSAKYRLQGTIPRGDVSLTILNPSESDSGVYCCRIEVPGWFNDVKINVRLNLQRASTTTHRTATTTTRRTTTTSPTTTRQMTTTPAALPTTVVTTPDLTTGTPLQMTTIAVFTTANTCLSLTPSTLPEEATGLLTPEPSKEGPILTAESETVLPSDSWSSVESTSADTVLLTSKESKVWDLPSTSHVSMWKTSDSVSSPQPGASDTAVPEQNKTTKTGQMDGIPMSMKNEMPISQLLMIIAPSLGFVLFALFVAFLLRGKLMETYCSQKHTRLDYIGDSKNVLNDVQHGREDEDGLFTL

SEQ ID NO: 42 - protein, 43 aa, Artificial Sequence
  The cytoplasmic domain of human TIMD4
LRGKLMETYCSQKHTRLDYIGDSKNVLNDVQHGREDEDGLFTL

SEQ ID NO: 43 - protein, 21 aa, Artificial Sequence
  The transmembrane domain of human TIMD4
LLMIIAPSLGFVLFALFVAFL

SEQ ID NO: 44 - protein, 290 aa, Artificial Sequence [sequence omitted]

SEQ ID NO: 45 - protein, 343 aa, Mus musculus
  misc_feature: Mouse TIMD4
MSKGLLLLWLVTELWWLYLTPAASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQTILIIACCVGFVLMVLLFLAFLLRGKVTGANCLQRHKRPDNTEDSDSVLNDMSHGRDDEDGIFTL

SEQ ID NO: 46 - protein, 43 aa, Artificial Sequence
  The cytoplasmic domain of mouse TIMD4
LRGKVTGANCLQRHKRPDNTEDSDSVLNDMSHGRDDEDGIFTL

SEQ ID NO: 47 - protein, 257 aa, Artificial Sequence
  The extracellular domain of mouse TIMD4
ASEDTIIGFLGQPVTLPCHYLSWSQSRNSMCWGKGSCPNSKCNAELLRTDGTRIISRKSTKYTLLGKVQFGEVSLTISNTNRGDSGVYCCRIEVPGWFNDVKKNVRLELRRATTTKKPTTTTRPTTTPYVTTTTPELLPTTVMTTSVLPTTTPPQTLATTAFSTAVTTCPSTTPGSFSQETTKGSAFTTESETLPASNHSQRSMMTISTDIAVLRPTGSNPGILPSTSQLTTQKTTLTTSESLQKTTKSHQINSRQT

SEQ ID NO: 48 - protein, 1140 aa, Homo sapiens
  misc_feature: Human Megf10
MVISLNSCLSFICLLLCHWIGTASPLNLEDPNVCSHWESYSVTVQESYPHPFDQIYYTSCTDILNWFKCTRHRVSYRTAYRHGEKTMYRRKSQCCPGFYESGEMCVPHCADKCVHGRCIAPNTCQCEPGWGGTNCSSACDGDHWGPHCTSRCQCKNGALCNPITGACHCAAGFRGWRCEDRCEQGTYGNDCHQRCQCQNGATCDHVTGECRCPPGYTGAFCEDLCPPGKHGPQCEQRCPCQNGGVCHHVTGECSCPSGWMGTVCGQPCPEGRFGKNCSQECQCHNGGTCDAATGQCHCSPGYTGERCQDECPVGTYGVLCAETCQCVNGGKCYHVSGACLCEAGFAGERCEARLCPEGLYGIKCDKRCPCHLENTHSCHPMSGECACKPGWSGLYCNETCSPGFYGEACQQICSCQNGADCDSVTGKCTCAPGFKGIDCSTPCPLGTYGINCSSRCGCKNDAVCSPVDGSCTCKAGWHGVDCSIRCPSGTWGFGCNLTCQCLNGGACNTLDGTCTCAPGWRGEKCELPCQDGTYGLNCAERCDCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGICTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPSGTYGYGCRQICDCLNNSTCDHITGTCYCSPGWKGARCDQAGVIIVGNLNSLSRTSTALPADSYQIGAIAGIIILVLVVLFLLALFIIYRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 49 - protein, 262 aa, Artificial Sequence
  The cytoplasmic domain of human Megf10
YRHKQKGKESSMPAVTYTPAMRVVNADYTISGTLPHSNGGNANSHYFTNPSYHTLTQCATSPHVNNRDRMTVTKSKNNQLFVNLKNVNPGKRGPVGDCTGTLPADWKHGGYLNELGAFGLDRSYMGKSLKDLGKNSEYNSSNCSLSSSENPYATIKDPPVLIPKSSECGYVEMKSPARRDSPYAEINNSTSANRNVYEVEPTVSVVQGVFSNNGRLSQDPYDLPKNSHIPCHYDLLPVRDSSSSPKQEDSGGSSSNSSSSSE

SEQ ID NO: 50 - protein, 21 aa, Artificial Sequence
  The transmembrane domain of human Megf10
AIAGIIILVLVVLFLLALFII

SEQ ID NO: 51 - protein, 832 aa, Artificial Sequence
  The extracellular domain of human Megf10
LNLEDPNVCSHWESYSVTVQESYPHPFDQIYYTSCTDILNWFKCTRHRVSYRTAYRHGEKTMYRRKSQCCPGFYESGEMCVPHCADKCVHGRCIAPNTCQCEPGWGGTNCSSACDGDHWGPHCTSRCQCKNGALCNPITGACHCAAGFRGWRCEDRCEQGTYGNDCHQRCQCQNGATCDHVTGECRCPPGYTGAFCEDLCPPGKHGPQCEQRCPCQNGGVCHHVTGECSCPSGWMGTVCGQPCPEGRFGKNCSQECQCHNGGTCDAATGQCHCSPGYTGERCQDECPVGTYGVLCAETCQCVNGGKCYHVSGACLCEAGFAGERCEARLCPEGLYGIKCDKRCPCHLENTHSCHPMSGECACKPGWSGLYCNETCSPGFYGEACQQICSCQNGADCDSVTGKCTCAPGFKGIDCSTPCPLGTYGINCSSRCGCKNDAVCSPVDGSCTCKAGWHGVDCSIRCPSGTWGFGCNLTCQCLNGGACNTLDGTCTCAPGWRGEKCELPCQDGTYGLNCAERCDCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGICTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPSGTYGYGCRQICDCLNNSTCDHITGTCYCSPGWKGARCDQAGVIIVGNLNSLSRTSTALPADSYQIG

SEQ ID NO: 52 - protein, 1147 aa, Mus musculus
  misc_feature: Mouse Megf10
MAISSSSCLGLICSLLCHWVGTASSLNLEDPNVCSHWESYSVTVQESYPHPFDQIYYTSCTDILNWFKCTRHRISYRTAYRHGEKTMYRRKSQCCPGFYESRDMCVPHCADKCVHGRCIAPNTCQCEPGWGGTNCSSACDGDHWGPHCSSRCQCKNRALCNPITGACHCAAGYRGWRCEDRCEQGTYGNDCHQRCQCQNGATCDHITGECRCSPGYTGAFCEDLCPPGKHGPHCEQRCPCQNGGVCHHVTGECSCPSGWMGTVCGQPCPEGRFGKNCSQECQCHNGGTCDAATGQCHCSPGYTGERCQDECPVGSYGVRCAEACRCVNGGKCYHVSGTCLCEAGFSGELCEARLCPEGLYGIKCDKRCPCHLDNTHSCHPMSGECGCKPGWSGLYCNETCSPGFYGEACQQICSCQNGADCDSVTGRCACAPGFKGTDCSTPCPLGRYGINCSSRCGCKNDAVCSPVDGSCICKAGWHGVDCSIRCPSGTWGFGCNLTCQCLNGGACNTLDGTCTCAPGWRGAKCEFPCQDGTYGLNCAERCDCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGVCTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPAGTYGYGCRQICDCLNNSTCDHITGTCYCSPGWKGARCDQAGVIIVGNLNSLSRTSTALPADSYQIGAIAGIVVLVLVVLFLLALFIIYRHKQKRKESSMPAVTYTPAMRVINADYTIAETLPHSNGGNANSHYFTNPSYHTLSQCATSPHVNNRDRMTIAKSKNNQLFVNLKNVNPGKRGTLVDCTGTLPADWKQGGYLNELGAFGLDRSYMGKSLKDLGKNSEYNSSTCSLSSSENPYATIKDPPALLPKSSECGYVEMKSPARRDSPYAEINNSTPANRNVYEVEPTVSVVQGVFSNSGHVTQDPYDLPKNSHIPCHYDLLPVRDSSSSPKREDGGGSNSTSSNSTSSSSSSSE

SEQ ID NO: 53 - protein, 269 aa, Artificial Sequence
  The cytoplasmic domain of mouse Megf10
YRHKQKRKESSMPAVTYTPAMRVINADYTIAETLPHSNGGNANSHYFTNPSYHTLSQCATSPHVNNRDRMTIAKSKNNQLFVNLKNVNPGKRGTLVDCTGTLPADWKQGGYLNELGAFGLDRSYMGKSLKDLGKNSEYNSSTCSLSSSENPYATIKDPPALLPKSSECGYVEMKSPARRDSPYAEINNSTPANRNVYEVEPTVSVVQGVFSNSGHVTQDPYDLPKNSHIPCHYDLLPVRDSSSSPKREDGGGSNSTSSNSTSSSSSSSE

SEQ ID NO: 54 - protein, 21 aa, Artificial Sequence
  The transmembrane domain of mouse Megf10
AIAGIVVLVLVVLFLLALFII

SEQ ID NO: 55 - protein, 832 aa, Artificial Sequence
  The extracellular domain of mouse Megf10
LNLEDPNVCSHWESYSVTVQESYPHPFDQIYYTSCTDILNWFKCTRHRISYRTAYRHGEKTMYRRKSQCCPGFYESRDMCVPHCADKCVHGRCIAPNTCQCEPGWGGTNCSSACDGDHWGPHCSSRCQCKNRALCNPITGACHCAAGYRGWRCEDRCEQGTYGNDCHQRCQCQNGATCDHITGECRCSPGYTGAFCEDLCPPGKHGPHCEQRCPCQNGGVCHHVTGECSCPSGWMGTVCGQPCPEGRFGKNCSQECQCHNGGTCDAATGQCHCSPGYTGERCQDECPVGSYGVRCAEACRCVNGGKCYHVSGTCLCEAGFSGELCEARLCPEGLYGIKCDKRCPCHLDNTHSCHPMSGECGCKPGWSGLYCNETCSPGFYGEACQQICSCQNGADCDSVTGRCACAPGFKGTDCSTPCPLGRYGINCSSRCGCKNDAVCSPVDGSCICKAGWHGVDCSIRCPSGTWGFGCNLTCQCLNGGACNTLDGTCTCAPGWRGAKCEFPCQDGTYGLNCAERCDCSHADGCHPTTGHCRCLPGWSGVHCDSVCAEGRWGPNCSLPCYCKNGASCSPDDGICECAPGFRGTTCQRICSPGFYGHRCSQTCPQCVHSSGPCHHITGLCDCLPGFTGALCNEVCPSGRFGKNCAGVCTCTNNGTCNPIDRSCQCYPGWIGSDCSQPCPPAHWGPNCIHTCNCHNGAFCSAYDGECKCTPGWTGLYCTQRCPLGFYGKDCALICQCQNGADCDHISGQCTCRTGFMGRHCEQKCPAGTYGYGCRQICDCLNNSTCDHITGTCYCSPGWKGARCDQAGVIIVGNLNSLSRTSTALPADSYQIG

SEQ ID NO: 56 - protein, 164 aa, Homo sapiens
  misc_feature: Human CD3 zeta chain
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - protein, 113 aa, Artificial Sequence
  The cytoplasmic domain of human CD3 zeta chain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 58 - protein, 21 aa, Artificial Sequence
  The transmembrane domain of human CD3 zeta chain
LCYLLDGILFIYGVILTALFL

SEQ ID NO: 59 - protein, 9 aa, Artificial Sequence
  The extracellular domain of human CD3 zeta chain
QSFGLLDPK

SEQ ID NO: 60 - protein, 164 aa, Mus musculus
  misc_feature: Mouse CD3 zeta chain
MKWKVSVLACILHVRFPGAEAQSFGLLDPKLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 61 - protein, 113 aa, Artificial Sequence
  The cytoplasmic domain of mouse CD3 zeta chain
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 62 - protein, 21 aa, Artificial Sequence
  The transmembrane domain of mouse CD3 zeta chain
LCYLLDGILFIYGVIITALYL

SEQ ID NO: 63 - protein, 9 aa, Artificial Sequence
  The extracellular domain of mouse CD3 zeta chain
QSFGLLDPK

SEQ ID NO: 64 - protein, 760 aa, Homo sapiens
  misc_feature: Human transferrin receptor
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 65 - protein, 67 aa, Artificial Sequence
  The cytoplasmic domain of human transferrin receptor
MMDQARSAFSNLFGGEPLSYTRFSLARQVDGDNSHVEMKLAVDEEENADNNTKANVTKPKRCSGSIC

SEQ ID NO: 66 - protein, 21 aa, Artificial Sequence
  The transmembrane domain of human transferrin receptor
YGTIAVIVFFLIGFMIGYLGY

SEQ ID NO: 67 - protein, 672 aa, Artificial Sequence
  The extracellular domain of human transferrin receptor
CKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 68 - protein, 202 aa, Artificial Sequence
  The extracellular domain of human transferrin receptor
EDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 69 - protein, 229 aa, Homo sapiens
  misc_feature: Human Clec-2 receptor
MQDEDGYITLNIKTRKPALISVGSASSSWWRVMALILLILCVGMVVGLVALGIWSVMQRNYLQGENENRTGTLQQLAKRFCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTFCENKHYLMCERKAGMTKVDQLP

SEQ ID NO: 70 - protein, 33 aa, Artificial Sequence
  The cytoplasmic domain of human Clec-2 receptor
MQDEDGYITLNIKTRKPALISVGSASSSWWRVM

SEQ ID NO: 71 - protein, 21 aa, Artificial Sequence
  The transmembrane domain of human Clec-2 receptor
ALILLILCVGMVVGLVALGIW

SEQ ID NO: 72 - protein, 175 aa, Artificial Sequence
  The extracellular domain of human Clec-2 receptor
SVMQRNYLQGENENRTGTLQQLAKRFCQYVVKQSELKGTFKGHKCSPCDTNWRYYGDSCYGFFRHNLTWEESKQYCTDMNATLLKIDNRNIVEYIKARTHLIRWVGLSRQKSNEVWKWEDGSVISENMFEFLEDGKGNMNCAYFHNGKMHPTFCENKHYLMCERKAGMTKVDQLP

SEQ ID NO: 73 - protein, 231 aa, Homo sapiens
  misc_feature: Human KLRF1 receptor
MQDEERYMTLNVQSKKRSSAQTSQLTFKDYSVTLHWYKILLGISGTVNGILTLTLISLILLVSQGVLLKCQKGSCSNATQYEDTGDLKVNNGTRRNISNKDLCASRSADQTVLCQSEWLKYQGKCYWFSNEMKSWSDSYVYCLERKSHLLIIHDQLEMAFIQKNLRQLNYVWIGLNFTSLKMTWTWVDGSPIDSKIFFIKGPAKENSCAAIKESKIFSETCSSVFKWICQY

SEQ ID NO: 74 - protein, 38 aa, Artificial Sequence
  The cytoplasmic domain of human KLRF1 receptor
MQDEERYMTLNVQSKKRSSAQTSQLTFKDYSVTLHWYK

SEQ ID NO: 75 - protein, 21 aa, Artificial Sequence
  The transmembrane domain of human KLRF1 receptor
ILLGISGTVNGILTLTLISLI

SEQ ID NO: 76 - protein, 172 aa, Artificial Sequence
  The extracellular domain of human KLRF1 receptor
LLVSQGVLLKCQKGSCSNATQYEDTGDLKVNNGTRRNISNKDLCASRSADQTVLCQSEWLKYQGKCYWFSNEMKSWSDSYVYCLERKSHLLIIHDQLEMAFIQKNLRQLNYVWIGLNFTSLKMTWTWVDGSPIDSKIFFIKGPAKENSCAAIKESKIFSETCSSVFKWICQY

SEQ ID NO: 77 - protein, 247 aa, Homo sapiens
  misc_feature: Human Dectin-1 receptor
MEYHPDLENLDEDGYTQLHFDSQSNTRIAVVSEKGSCAASPPWRLIAVILGILCLVILVIAVVLGTMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 78 - protein, 44 aa, Artificial Sequence
  The cytoplasmic domain of human Dectin-1 receptor
MEYHPDLENLDEDGYTQLHFDSQSNTRIAVVSEKGSCAASPPWR

SEQ ID NO: 79 - protein, 21 aa, Artificial Sequence
  The transmembrane domain of human Dectin-1 receptor
LIAVILGILCLVILVIAVVLG

SEQ ID NO: 80 - protein, 182 aa, Artificial Sequence
  The extracellular domain of human Dectin-1 receptor
TMAIWRSNSGSNTLENGYFLSRNKENHSQPTQSSLEDSVTPTKAVKTTGVLSSPCPPNWIIYEKSCYLFSMSLNSWDGSKRQCWQLGSNLLKIDSSNELGFIVKQVSSQPDNSFWIGLSRPQTEVPWLWEDGSTFSSNLFQIRTTATQENPSPNCVWIHVSVIYDQLCSVPSYSICEKKFSM

SEQ ID NO: 81 - protein, 12 aa, Artificial Sequence
  chimeric protein
  misc_feature (4)..(5): Xaa can be any naturally occurring amino acid
  misc_feature (7)..(8): Xaa can be any naturally occurring amino acid
HAEXXYXXLQWD

SEQ ID NO: 82 - protein, 12 aa, Artificial Sequence
  chimeric protein
  misc_feature (4)..(5): Xaa can be any naturally occurring amino acid
  misc_feature (7)..(8): Xaa can be any naturally occurring amino acid
HEEXXYXXLQWD

SEQ ID NO: 83 - protein, 10 aa, Artificial Sequence
  chimeric protein
  misc_feature (5)..(6): Xaa can be any naturally occurring amino acid
  misc_feature (8)..(9): Xaa can be any naturally occurring amino acid
MHAEXXYXXL

SEQ ID NO: 84 - protein, 10 aa, Artificial Sequence
  chimeric protein
  misc_feature (5)..(6): Xaa can be any naturally occurring amino acid
  misc_feature (8)..(9): Xaa can be any naturally occurring amino acid
MHEEXXYXXL

SEQ ID NO: 85 - protein, 9 aa, Artificial Sequence
  chimeric protein
  misc_feature (4)..(5): Xaa can be any naturally occurring amino acid
  misc_feature (7)..(8): Xaa can be any naturally occurring amino acid
HAEXXYXXL

SEQ ID NO: 86 - protein, 9 aa, Artificial Sequence
  chimeric protein
  misc_feature (4)..(5): Xaa can be any naturally occurring amino acid
  misc_feature (7)..(8): Xaa can be any naturally occurring amino acid
HEEXXYXXL

SEQ ID NO: 87 - protein, 9 aa, Artificial Sequence
  chimeric protein
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
HXEXXYXXL

SEQ ID NO: 88 - protein, 21 aa, Artificial Sequence
  The transmembrane domain of mouse TIMD4
ILIIACCVGFVLMVLLFLAFL

SEQ ID NO: 89 - protein, 13 aa, Artificial Sequence
  The signalling portion of human DNGR-1 cytoplasmic domain
MHEEEIYTSLQWD

SEQ ID NO: 90 - protein, 13 aa, Artificial Sequence
  Syk binding sequence
  misc_feature (5)..(6): Xaa can be any naturally occurring amino acid
  misc_feature (8)..(9): Xaa can be any naturally occurring amino acid
MHEEXXYXXLQWD

SEQ ID NO: 91 - protein, 13 aa, Artificial Sequence
  Syk-binding sequence
  misc_feature (3)..(3): Xaa can be any naturally occurring amino acid
MHXEEIYTSLQWD

SEQ ID NO: 92 - protein, 7 aa, Artificial Sequence
  linker sequence
GGGSGGG

SEQ ID NO: 93 - protein, 10 aa, Artificial Sequence
  linker sequence
GGGGSGGGGS

SEQ ID NO: 94 - protein, 4 aa, Artificial Sequence
  linker sequence
PGPG

SEQ ID NO: 95 - protein, 12 aa, Artificial Sequence
  linker sequence
GSAGSAAGSGEF

SEQ ID NO: 96 - protein, 226 aa, Artificial Sequence
  chimeric receptor
MHAEEIYTSLQWDIPTSEASQKCQSPSKCSGAVGLGILCFVVVVVAAVLGALAFWRHNSGRNPEEKDSFLSRNKENHKPTESSLDEKVAPSKASQTTGGFSQSCLPNWIMHGKSCYLFSFSGNSWYGSKRHCSQLGAHLLKIDNSKEFEFIESQTSSHRINAFWIGLSRNQSEGPWFWEDGSAFFPNSFQVRNAVPQESLLHNCVWIHGSEVYNQICNTSSYSICE

SEQ ID NO: 97 - protein, 728 aa, Artificial Sequence
  chimeric receptor
MHEEEIYTSLQWDSPAPDTYQKCLSSNKCSGACCLVMVISCVFCMGLLTASIFLGVCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 98 - protein, 340 aa, Artificial Sequence
  chimeric receptor
MILTSFGDDMWLLTTLLLWVPVGGEVVNATKAVITLQPPWVSIFQKENVTLWCEGPHLPGDSSTQWFINGTAVQISTPSYSIPEASFQDSGEYRCQIGSSMPSDPVQLQIHNDWLLLQASRRVLTEGEPLALRCHGWKNKLVYNVVFYRNGKSFQFSSDSEVAILKTNLSHSGIYHCSGTGRHRYTSAGVSITVKELFTTPVLRASVSSPFPEGSLVTLNCETNLLLQRPGLQLHFSFYVGSKILEYRNTSSEYHIARAEREDAGFYWCEVATEDSSVLKRSPELELQVLGPQSSAPVWFHILFYLSVGIMFSLNTVLYVGGGSGGGMHEEEIYTSLQWD

(19) United States
(12) Patent Application Publication
(10) Pub. No.: US 2024/0100161 A1
(43) Pub. Date: Mar. 28, 2024

(54) CYTOSOLIC DELIVERY

(71) Applicant: THE FRANCIS CRICK INSTITUTE LIMITED, London, Greater London (GB)

(72) Inventors: Caetano REIS E SOUSA, London, Greater London (GB); Johnathan Anias CANTON, Calgary (CA); Conor Maurice HENRY, London, Greater London (GB); Hanna BLEES, Oberursel (DE)

(21) Appl. No.: 18/018,685

(22) PCT Filed: Jul. 30, 2021
(86) PCT No.: PCT/EP2021/071399

§ 371 Date: Jan. 30, 2023

(30) Foreign Application Priority Data
Jul. 30, 2020 (GB) 2011859.2

(51) Int. Cl. A61K 39/00; C07K 14/705; C07K 16/40

(52) U.S. Cl. CPC A61K 39/4633; A61K 39/4614; A61K 39/4622; A61K 39/4644; C07K 14/7056; C07K 16/40; C07K 2319/03

(57) ABSTRACT

The present invention relates to chimeric receptors capable of facilitating cross-presentation (XP) of antigens, and methods of doing the same.

### REFERENCE TO AN ELECTRONIC SEQUENCE LISTING

The content of the electronic sequence listing submitted on Aug. 4, 2023, as a text file named “10480-011US1_ST25”, which was created on Aug. 3, 2023, and which has a size of 144,147 bytes, is hereby incorporated by reference in its entirety pursuant to 37 CFR 1.52(e)(5).

### FIELD OF THE INVENTION

The present invention relates to means for delivering functional macromolecules to the cytosol. The invention provides chimeric receptors comprising parts of DNGR-1, a transmembrane receptor, as well as cells expressing the chimeric receptors, means for producing the chimeric receptors, and medical uses thereof. Functional macromolecules, such as biopolymers, which bind and trigger DNGR-1 and/or the chimeric receptors of the invention, and constructs comprising said macromolecules, are also provided.

### BACKGROUND

DNGR-1 (also known as CLEC9A) is a C-type lectin that has been previously described as a key mediator of cross-presentation by type 1 conventional dendritic cells (cDC1s) (D Sancho et al. Nature 2009¹²; WO2009/013484A1²⁰, both of which are specifically incorporated by reference herein). Cross-presentation (XP) refers to a process, performed by antigen-presenting cells (APCs), of presenting exogenous antigens on MHC class I molecules to cytotoxic T lymphocytes (CTL). XP is essential for the induction of protective CTL responses against tumours and many viruses¹⁻⁷.

Macrophages, monocyte-derived dendritic cells and other myeloid cell types, as well as non-immune cells, have been used extensively to dissect some of the mechanisms involved in XP^{8,21,38}. While this has led to the view that XP in many cases involves the cytosolic pathway (phagosome to cytosolic transfer; P2C), these studies have generally fallen short of explaining how P2C occurs, especially for complex substrates such as dead cells. Indeed, the actual mechanism underlying the regulation of cross-presentation of antigens from cellular corpses by DNGR-1 is reported to remain a mystery²⁸. XP in vivo, notably in the context of virus infection and anti-tumour immunity, is abrogated in cDC1-deficient Batf3^{-/-} mice³⁹, pointing to cDC1 as a non-redundant cross-presenting APC.

Relatively few papers have focused on XP mechanisms specifically in cDC1s^{7,30,36,40-42}. Typically, dying virally-infected or tumour cells are thought to be sources of exogenous antigen for cross-presenting cDC1s. The DNGR-1 expressed by these cells detects F-actin/myosin complexes exposed on dead cell debris and promotes XP of corpse-associated antigens¹²⁻¹⁶, thus coupling the detection of cell death with immunity.

A tyrosine-containing hemITAM motif in the cytoplasmic tail of DNGR-1⁵⁴ allows binding of the tyrosine kinase, Syk, upon tyrosine phosphorylation¹². However, until now, the subsequent intracellular signalling events that allow DNGR-1 to facilitate the XP of bound antigens (and the mechanisms underlying XP in general) have remained poorly understood. Some previous disclosures have examined DNGR-1 function by generating chimeric proteins comprising the extracellular domain of DNGR-1 fused to the intracellular domain of CD3ζ^{12,20}. In this context, detectable signalling is triggered when a bivalent ligand binds the DNGR-1 extracellular domain, causing the readily detectable signalling cascade initiated by CD3ζ activation. However, the lack of understanding of the cellular mechanisms responsible for XP has inhibited research into whether DNGR-1 could provide the basis of useful biomedical technologies.

### SUMMARY OF THE INVENTION

The inventors have uncovered the mechanism by which DNGR-1 mediates cross presentation (XP). This led the inventors to provide the chimeric receptors of the invention, which provides a platform technology to facilitate XP of a target antigen (which is not limited to any particular type or class of antigen). Surprisingly, this can be applied to achieve XP of the target antigen by a range of cell types, not just professional antigen presenting cells (APCs). This new understanding of the XP pathway also leads to the provision of a way of delivering macromolecules such as biopolymers to the cytosol without degradation. The invention and its underlying mechanism are explained in more detail below.

The inventors found that DNGR-1 dependent XP proceeds via a cytosolic pathway. DNGR-1 promotes phagosomal rupture, which allows internalised antigens to be released into the cytosol where they are processed and presented via the conventional MHC class I antigen processing pathway. The inventors surprisingly found that the internalised antigens are not substantially degraded before being released into the cytosol. Using chimeric receptors, the inventors have now demonstrated that the cytoplasmic tail of DNGR-1 is a key mediator of this process, thus enabling XP. The inventors show that this requires only the DNGR-1 signalling domain, which recruits and activates spleen tyrosine kinase (Syk) and NADPH oxidase to cause lipid peroxidation and phagosomal membrane instability. Notably, DNGR-1 signalling can induce phagosomal membrane rupture and XP in heterologous cells, including non-professional APCs. These results show that phagosomal rupture is coupled to XP, providing a simple mechanism for access of exogenous antigens to the endogenous MHC I pathway. This mechanism is non-selective and does not require specific antigen transporters. Furthermore, the basic machinery for phagosomal rupture is not limited to DCs, thus other non-APCs have the potential to cross-present exogenous antigens. However, the effective engagement of this machinery requires a dedicated XP signalling receptor such as DNGR-1.

Thus, the invention provides chimeric proteins comprising the signalling domain of the cytoplasmic tail of DNGR-1. The chimeric proteins of the invention can facilitate XP. By engineering cells (not limited to DCs) to express the chimeric proteins, the invention provides a wide range of cells that can recognise and process a desired target (not limited to the actin/myosin II signal on dead cells), and present antigens from the target to CD8+ T cells, to elicit a robust immune response to the target. The invention also provides a way of delivering biopolymers (such as proteins and nucleic acids) to the cytosol without being degraded.

Accordingly, in a first aspect, the invention provides a method of delivering a biopolymer to the cytosol of a cell, wherein the cell expresses a transmembrane protein comprising an intracellular domain that comprises a Syk-binding sequence derived from the signalling domain of the cytoplasmic tail of DNGR-1, wherein the biopolymer comprises a binding domain that can specifically bind an extracellular portion of the transmembrane protein, and wherein the method comprises contacting the cell with the biopolymer to allow the binding domain to bind to the extracellular portion of the transmembrane protein such that the biopolymer is internalised and translocated to the cytosol without being degraded in a phagosome. Preferably, the biopolymer further comprises a nucleic acid that encodes a gene product.

Thus, the invention provides a way of delivering payloads to the cytosol. The payload may be the biopolymer itself and/or an additional moiety, such as a nucleic acid encoding a gene product, that is covalently or non-covalently associated with the biopolymer. The delivery of such payloads finds use in research, diagnostic and medical applications. As such, the invention provides the biopolymer defined herein for use in medical methods, such as vaccination. In vitro methods are particularly suited to research and/or diagnostic applications.

In some embodiments, the biopolymer comprises a second domain, which does not directly bind to the extracellular portion of the transmembrane protein. This second domain is covalently joined to the binding domain, e.g. via a linker. In some embodiments, the linker can be cleaved by a protease present in the cytosol of the cell, allowing the second domain to dissociate from the binding domain in the cytosol. (The second biopolymer domain may be referred to as a payload.) The second domain may be a nucleic acid that encodes a gene product. The gene product may be a pro-apoptotic protein, an enzyme, or a cytotoxic peptide as described herein.

In some embodiments, the (first) biopolymer (that comprises a binding domain) is non-covalently associated with a second biopolymer. This second biopolymer may be referred to as a ‘second domain’ herein, and may also be referred to as a payload. A non-covalent association can be achieved e.g. by fusing the first biopolymer to an avidin or streptavidin moiety, and covalently linking a biotin ‘tag’ to the second biopolymer, or vice-versa. The avidin/streptavidin non-covalently binds the biotin tag, thus associating the first and second biopolymers with each other.

Thus, the binding domain of the biopolymer may be coupled to a second biopolymer domain, wherein the coupling may be covalent or non-covalent.

Polypeptide biopolymers, polynucleotide biopolymers and polysaccharide biopolymers are all envisaged.

The biopolymer may be a polypeptide and the binding domain may comprise at least part of the antigen-binding fragment of an antibody. For instance, the polypeptide may comprise an antibody V_{H} domain; and this can pair with an antibody V_{L} domain that is provided as a separate polypeptide. Alternatively, the polypeptide may comprise an antibody V_{L} domain; and this can pair with an antibody V_{H} domain that is provided as a separate polypeptide. In some embodiments, the polypeptide may comprise both antibody V_{H} and V_{L} domains, present on a single polypeptide chain, e.g. in the scFV format.

The biopolymer may be a nucleic acid and the binding domain may be an aptamer.

Combinations of biopolymers of different classes, for the binding domain and for the second domain, are also envisaged. For instance, a binding domain that is an antibody (a polypeptide) may be coupled to a non-peptide second domain. Such coupling of biopolymer domains of different classes can be achieved covalently or non-covalently, e.g. as described herein. The term “biopolymer” used herein, and the methods of delivering said biopolymer, are intended to encompass biopolymer pairings where the binding domain is one type of biopolymer and the payload is another type of biopolymer.

The biopolymer may comprise multiple binding domains. For instance, two or more antibodies and one or more second biopolymer domains can all be immobilised on a single carrier particle, e.g. a latex bead.

In embodiments where the biopolymer comprises a polynucleotide, the polynucleotide may encode a gene product and is capable of expressing the gene product in a host cell. The encoded gene product may be a polypeptide disclosed herein. In other embodiments, the polynucleotide is, or encodes, an RNA sequence that interferes with the expression of another protein in the cell (e.g. an RNAi molecule such as an siRNA). The polynucleotide may be DNA. The DNA may be capable of activating the STING pathway. The polynucleotide may be RNA. The RNA may be capable of activating the RIG-I and/or MDA5 pathways.

In some embodiments, the second domain comprises a cytotoxin or pro-apoptotic protein. For instance, the second domain may comprise cytochrome C, a caspase, a maytansinoid, a dolastatin, an auristatin drug analogue, a cryptophycin, a duocarmycin deriative, an enediyne antibiotic, or a pyrolobenodiazepine. In some embodiments, the second domain comprises an antibody that binds to an intracellular target. In some embodiments, the biopolymer comprises a tumour antigen (second domain) conjugated to an anti-DNGR-1 antibody (first domain). In some embodiments, the second domain is not a peptide antigen. In other words, in some embodiments, the second domain is a whole protein or a whole domain of a protein. In some embodiments, the second domain comprises more than 50 amino acid residues.

In some embodiments, the second domain is a nucleic acid that encodes a cytotoxin or pro-apoptotic protein. For instance, the second domain may encode cytochrome C, a caspase, a maytansinoid, a dolastatin, an auristatin drug analogue, a cryptophycin, a duocarmycin deriative, an enediyne antibiotic, or a pyrolobenodiazepine. In some embodiments, the second domain encodes an antibody that binds to an intracellular target. In some embodiments, the biopolymer encodes a tumour antigen (second domain) conjugated to an anti-DNGR-1 antibody (first domain). In some embodiments, the second domain is not a peptide antigen. In other words, in some embodiments, the second domain is a whole protein or a whole domain of a protein. In some embodiments, the second domain encodes more than 50 amino acid residues.

The one or more binding domains and/or second domains of the biopolymer may be linked to each other via a linker, e.g. a peptide linker. Peptide linkers are well known in the art. The skilled person can choose from a selection of linker sequences such as GGGSGGG (SEQ ID NO:92), GGGGSGGGGS (SEQ ID NO:93), PGPG (SEQ ID NO:94) and GSAGSAAGSGEF (SEQ ID NO:95). Combinations and repeats of these linker sequences may also be used.

The cell may be a tumour cell. The cell may be an immune cell. In some embodiments, the cell expresses an NADPH oxidase comprising NOX2.

The transmembrane protein expressed by the cell of the first aspect may be a chimeric receptor of the invention, as described in more detail elsewhere herein. The method of the first aspect may comprise the step of expressing the transmembrane protein in the cell before the cell is contacted with the biopolymer.

The method of expression may comprise delivering a vector comprising a nucleic acid to the cell. The vector may be a non-viral gene therapy vector, e.g. a plasmid (optionally complexed with a liposome or delivery agent) or the vector may be a viral gene therapy vector, e.g. a viral vector. Lentiviral vectors, adenoviral vectors and adenovirus associated virus (AAV) based vectors are all envisaged.

In other embodiments, the transmembrane protein expressed by the cell is DNGR-1. In these embodiments, the binding domain of the biopolymer will bind the extracellular domain of DNGR-1. For instance, the binding domain of the biopolymer may be a polypeptide comprising a DNGR-1 binding fragment of the F-actin/myosin complex. Alternatively, the binding domain of the biopolymer may be an antibody or aptamer that specifically binds DNGR-1.

In a second aspect, the invention provides a method of treating a disease in a patient in need thereof, by delivering a biopolymer to the cytosol of a cell of the patient using a method of the first aspect. In a related third aspect, the invention provides a biopolymer for use in a method of treating a disease in a patient in need thereof, the method comprising delivering a biopolymer to the cytosol of a cell of the patient using a method of the first aspect. In a related fourth aspect, the invention provides the use of a biopolymer in the manufacture of a medicament for treating a disease in in a patient in need thereof, the treatment comprising delivering a biopolymer to the cytosol of a cell of the patient using a method of the first aspect.

The biopolymer can be delivered to the patient's cell while it is still in the patient's body, by administering the biopolymer to the patient. The cell may be a cancer cell. The biopolymer may be administered by injection to the cancer or surrounding tissue. Alternatively, the biopolymer can be delivered to the patient's cell ex vivo, following a step of harvesting the cell. The cell may be an immune cell. In these embodiments, the cell can be reintroduced to the patient as a cell therapy.

In some embodiments, the biopolymer is administered to a cancer patient to elicit an anti-cancer Th1 response. In some embodiments, the biopolymer comprises an autoantigen and the biopolymer is administered to a patient who is suffering from an autoimmune disease, to elicit a tolerogenic response to the autoantigen.

In a fifth aspect, the invention provides a biopolymer as described herein. Nucleic acid molecules that encode polypeptide biopolymers of the invention are also provided. In some embodiments, these nucleic acids form part of a vector.

In a sixth aspect, the invention provides a chimeric receptor comprising an extracellular target binding domain, a transmembrane domain, and an intracellular domain that comprises a Syk-binding sequence derived from the signalling domain of the cytoplasmic tail of DNGR-1, wherein said Syk-binding sequence contains a tyrosine residue. The Syk-binding sequence may comprise a hemITAM. In some embodiments, the hemITAM comprises the amino acid sequence set forth in SEQ ID NO:14 (EXXYXXL; wherein X represents any amino acid residue).

In some embodiments, the Syk-binding sequence comprises an amino acid sequence as set forth in SEQ ID NO:15 (MHAEXXYXXLQWD), wherein ‘X’ represents any amino acid residue. In some embodiments, the Syk-binding sequence comprises an amino acid sequence as set forth in SEQ ID NO:15 (MHAEXXYXXLQWD), wherein ‘X’ represents any amino acid residue, and wherein one, two or all three of the residues at the N-terminus can be removed or substituted with another amino acid residue, and/or wherein one, two or all three of the residues at the C-terminus can be removed or substituted with another amino acid residue. In some embodiments, the Syk-binding sequence comprises an amino acid sequence as set forth in SEQ ID NO:90 (MHEEXXYXXLQWD). In some embodiments, the Syk-binding sequence comprises an amino acid sequence as set forth in SEQ ID NO:90 (MHEEXXYXXLQWD), wherein ‘X’ represents any amino acid residue, and wherein one, two or all three of the residues at the N-terminus can be removed or substituted with another amino acid residue, and/or wherein one, two or all three of the residues at the C-terminus can be removed or substituted with another amino acid residue. In some embodiments, the Syk-binding sequence comprises an amino acid sequence as set forth in SEQ ID NO:11 (MHAEEIYTSLQWD), optionally wherein one, two or three amino acid residues are substituted with another amino acid residue. Preferably, the N-terminal amino acid residue of the intracellular signalling domain lies at the N-terminus of the chimeric receptor of the invention. In some embodiments, the Syk-binding sequence comprises an amino acid sequence as set forth in SEQ ID NO:89 (MHEEEIYTSLQWD).

SEQ ID NO:11 is derived from mouse DNGR-1, whereas SEQ ID NO:69 is derived from human DNGR-1. These sequences differ from each other at the third amino acid residue, which is alanine in mouse and is glutamic acid in human. Thus, the Syk-binding sequence may comprises an amino acid sequence as set forth in SEQ ID NO:91, which is MHXEEIYTSLQWD, wherein X represents any amino acid. Preferably, the X in SEQ ID NO:91 is alanine (A) or glutamic acid (E).

In some embodiments, the target binding domain binds a target that is present on a pathogen, a pathogenic cell, a dead cell or a diseased cell. The target antigen may be present on a cancer cell, The target antigen may be a tumour antigen, e.g. a neoantigen. For instance, the tumour antigen may be CEA, ERBB2, EGFR, GD2, mesothelin, MUC1, PSMA, CAIX, CD133, c-Met, EGFR, EGFRvIII, Epcam, EphA2, FRα, CD19, CD20, GPC3, GUCY2C, HER1, HER2, ICAM-1, MAGE, or MET.

The target antigen may be present on a virally infected cell, e.g. at the cell surface, The target antigen may be a viral antigen, e.g. HCMV gB, influenza A hemagglutinin, influenza matrix 2 protein M2e, RSV glycoprotein F, SARS-Cov-2 Spike protein, HIV gp120 or HIV Env.

In some embodiments, the target binding domain of the chimeric receptor is derived from a non-DNGR-1 lectin, or is derived from a transferrin receptor. One example of a non-DNGR-1 lectin is Dectin-1. For instance, the target binding domain of the chimeric receptor can be derived from mouse Dectin-1.

In other embodiments, the target binding domain comprises an antibody variable region heavy chain (V_{H}) and/or variable region light chain (V_{L}). For instance, the chimeric receptor may comprise an antibody V_{H} domain; and this can pair with an antibody V_{L} domain that is expressed separately. Alternatively, the chimeric receptor may comprise an antibody V_{L} domain; and this can pair with an antibody V_{H} domain that is expressed separately. In some embodiments, the chimeric receptor may comprise both antibody V_{H} and V_{L} domains, present on a single polypeptide chain, e.g. in the single-chain variable fragment (scFv) format.

The target binding domain of the chimeric receptor may comprise the ligand-binding domain of a nucleic acid receptor (for instance a pattern recognition receptor), enabling the chimeric receptor to bind to a nucleic acid target and facilitating transportation of the nucleic acid to the cytosol.

The target binding domain, transmembrane domain and/or cytosolic domains of the chimeric receptor may be linked to each other via a linker, e.g. a peptide linker. Peptide linkers are well known in the art. The skilled person can choose from a selection of linker sequences such as GGGSGGG (SEQ ID NO:92), GGGGSGGGGS (SEQ ID NO:93), PGPG (SEQ ID NO:94) and GSAGSAAGSGEF (SEQ ID NO:95). Combinations and repeats of these linker sequences may also be used.

In some embodiments, the transmembrane domain of the chimeric receptor is from a type I transmembrane protein. In some embodiments, the extracellular domain of the chimeric receptor is from a type I transmembrane protein. In some embodiments, the transmembrane domain of the chimeric receptor is from a type II transmembrane protein. In some embodiments, the extracellular domain of the chimeric receptor is from a type II transmembrane protein.

A related aspect provides a cell comprising the chimeric receptor of the invention. The chimeric receptor may be expressed at the surface of a cell, i.e. at the cell membrane. The cell may be a non-professional APC, for instance a tumour cell. The cell may be an immune cell. The cell may be a professional APC, such as a macrophage or DC. In some embodiments, the cell expresses an NADPH oxidase comprising NOX2.

Disclosed herein are amino acid sequences of transmembrane receptors and their constituent domains. In view of the disclosed invention, it will be apparent that the intracellular domain that comprises a Syk-binding sequence (derived from the signalling domain of the cytoplasmic tail of DNGR-1) can be combined with the transmembrane domain and extracellular/ligand-binding domains from any of these exemplary sequences, to provide a broad range of chimeric receptors. This principle can be utilised to provide a chimeric receptor of the desired target specificity, to facilitate translocation of the target of interest to the cytosol of the cell.

Non-limiting, exemplary amino acid sequences of the chimeric receptors of the invention are provided here:

(SEQ ID NO: 96)
MHAEEIYTSLQWDIPTSEASQKCQSPSKCSGAVGLGILCF
VVVVVAAVLGALAFWRHNSGRNPEEKDSFLSRNKENHKPT
ESSLDEKVAPSKASQTTGGFSQSCLPNWIMHGKSCYLFSF
SGNSWYGSKRHCSQLGAHLLKIDNSKEFEFIESQTSSHRI
NAFWIGLSRNQSEGPWFWEDGSAFFPNSFQVRNAVPQESL
LHNCVWIHGSEVYNQICNTSSYSICE

The exemplary chimeric receptor of SEQ ID NO:96 consists of the cytoplasmic domain of mouse DNGR-1 (as set forth in SEQ ID NO:7) fused to the transmembrane and extracellular domains of mouse Dectin-1. SEQ ID NO:18 is the transmembrane domain. The extracellular domain comprises the neck region (SEQ ID NO:19) and the C-type lectin domain, CTLD (SEQ ID NO:20).

(SEQ ID NO: 97)
MHEEEIYTSLQWDSPAPDTYQKCLSSNKCSGACCLVMVIS
CVFCMGLLTASIFLGVCKGVEPKTECERLAGTESPVREEP
GEDFPAARRLYWDDLKRKLSEKLDSTDFTGTIKLLNENSY
VPREAGSQKDENLALYVENQFREFKLSKVWRDQHFVKIQV
KDSAQNSVIIVDKNGRLVYLVENPGGYVAYSKAATVTGKL
VHANFGTKKDFEDLYTPVNGSIVIVRAGKITFAEKVANAE
SLNAIGVLIYMDQTKFPIVNAELSFFGHAHLGTGDPYTPG
FPSFNHTQFPPSRSSGLPNIPVQTISRAAAEKLFGNMEGD
CPSDWKTDSTCRMVTSESKNVKLTVSNVLKEIKILNIFGV
IKGFVEPDHYVVVGAQRDAWGPGAAKSGVGTALLLKLAQM
FSDMVLKDGFQPSRSIIFASWSAGDFGSVGATEWLEGYLS
SLHLKAFTYINLDKAVLGTSNFKVSASPLLYTLIEKTMQN
VKHPVTGQFLYQDSNWASKVEKLTLDNAAFPFLAYSGIPA
VSFCFCEDTDYPYLGTTMDTYKELIERIPELNKVARAAAE
VAGQFVIKLTHDVELNLDYERYNSQLLSFVRDLNQYRADI
KEMGLSLQWLYSARGDFFRATSRLTTDFGNAEKTDRFVMK
KLNDRVMRVEYHFLSPYVSPKESPFRHVFWGSGSHTLPAL
LENLKLRKQNNGAFNETLFRNQLALATWTIQGAANALSGD
VWDIDNEF

The exemplary chimeric receptor of SEQ ID NO:97 consists of the cytoplasmic and transmembrane domains of human DNGR-1 (as set forth in SEQ ID NOs:2 and 3, respectively) fused to the extracellular domain of the human transferrin receptor, as set forth in SEQ ID NO:67.

(SEQ ID NO: 98)
MILTSFGDDMWLLTTLLLWVPVGGEVVNATKAVITLQPPW
VSIFQKENVTLWCEGPHLPGDSSTQWFINGTAVQISTPSY
SIPEASFQDSGEYRCQIGSSMPSDPVQLQIHNDWLLLQAS
RRVLTEGEPLALRCHGWKNKLVYNVVFYRNGKSFQFSSDS
EVAILKTNLSHSGIYHCSGTGRHRYTSAGVSITVKELFTT
PVLRASVSSPFPEGSLVTLNCETNLLLQRPGLQLHFSFYV
GSKILEYRNTSSEYHIARAEREDAGFYWCEVATEDSSVLK
RSPELELQVLGPQSSAPVWFHILFYLSVGIMFSLNTVLYV
GGGSGGGMHEEEIYTSLQWD

The exemplary chimeric receptor of SEQ ID NO:98 consists of the extracellular and transmembrane domains of mouse FcγRI (as set forth in SEQ ID NOs:36 and 35, respectively) fused to the Syk-binding sequence of human DNGR-1 (as set forth in SEQ ID NO:69), separated by a short linker sequence, GGGSGGG (SEQ ID NO92).

Peptide linkers are well known in the art. The skilled person can choose from a selection of linker sequences such as GGGSGGG (SEQ ID NO:92), GGGGSGGGGS (SEQ ID NO:93), PGPG (SEQ ID NO:94) and GSAGSAAGSGEF (SEQ ID NO:95). Combinations and repeats of these linker sequences may also be used.

In a further aspect, the invention provides nucleic acid molecules that encode the chimeric receptor of the invention. In some embodiments, this nucleic acid forms part of a vector.

The invention further provides host cells comprising the nucleic acid or vector that encodes the chimeric receptor. In preferred embodiments, the host cell expresses the chimeric receptor and is thus capable of cross-presenting an exogenous antigen that binds to the chimeric receptor.

In some embodiments, the cell that expresses the chimeric receptor cell is a myeloid cell, e.g. a macrophage, a monocyte, or a dendritic cell. In some embodiments, the cell that expresses the chimeric receptor is a lymphocyte. In some embodiments, the cell that expresses the chimeric receptor is not a professional antigen presenting cell. In some embodiments, the cell that expresses the chimeric receptor is not a dendritic cell.

The invention further provides a method of producing a cell that expresses the chimeric receptor of the invention, the method comprising:
a. providing a precursor cell;
b. introducing a nucleic acid or vector encoding the chimeric receptor into the precursor cell to produce a host cell; and
c. propagating the host cell of step b. under conditions that promote expression of the chimeric receptor encoded by said nucleic acid, such that the host cell expresses the chimeric receptor and thus becomes capable of cross-presenting the exogenous antigen.

In some embodiments of the invention, the exogenous antigen that is presented by the cell that expresses the chimeric receptor is the target that is bound by the target binding domain of the chimeric receptor. In other embodiments, the exogenous antigen that is presented by the cell that expresses the chimeric receptor is associated with the target that is bound by the target binding domain of the chimeric receptor.

The invention further provides pharmaceutical compositions comprising the vector of the invention. The invention also provides pharmaceutical compositions comprising the cell that expresses the chimeric receptor of the invention.

The pharmaceutical compositions of the invention are suitable for use in methods of treating cancer, where the method comprising administering the pharmaceutical composition to a cancer patient. The cancer may be a solid tumour and the method may optionally comprise injecting the pharmaceutical composition into the solid tumour, or into the tissue immediately surrounding the solid tumour. The pharmaceutical compositions of the invention may be used in medicine.

The pharmaceutical compositions of the invention are suitable for use in methods of treating infectious diseases, where the method comprises administering the pharmaceutical composition to the infected patient.

The pharmaceutical compositions of the invention are also suitable for use as vaccines or as part of vaccination programmes.

### DNGR-1 Binding Agents; DNGR-1 Binding Agent Complexes and Conjugates

The inventors have found that transmembrane receptors having an intracellular domain comprising the Syk-binding sequence of DNGR-1 can facilitate translocation of macromolecules to the cytosol, from the extracellular space, without sustaining substantial degradation. This provides a way of transporting functional macromolecules such as proteins and nucleic acids to the cytosol via targeting to the extracellular part of the transmembrane receptor. The transmembrane receptor may be wild type DNGR-1, or it may be a chimeric receptor of the invention. Thus the invention provides binding agents associated with a payload, wherein the binding agent is capable of binding the extracellular domain of the transmembrane receptor. This binding agent-payload complex/conjugate can access the cytosol of cells that express the transmembrane receptor.

The payload may be a biological macromolecule, for instance a protein or a nucleic acid. The payload may be associated with the binding agent via chemical conjugation (e.g. via expression as a fusion protein) or through high-affinity noncovalent interactions, such as that between biotin and streptavidin or avidin. The binding agent may be an anti-DNGR-1 antibody, a DNGR-1-binding aptamer, or a DNGR-1 binding fragment of the F-actin/myosin complex which is the natural ligand of DNGR-1. In some embodiments, the binding agent acts as a DNGR-1 agonist, to strongly stimulate the XP pathway described herein. In some embodiments, the payload is associated with multiple binding agents (e.g. two or more antibodies). Such “polyvalent” conjugates can be particularly effective at stimulating the XP pathway described herein.

This aspect of the invention is useful for delivering biologically active payloads into the cell, e.g. where the uncomplexed payload does not readily penetrate the cell membrane. Thus, in some embodiments, the payload is a protein or cytotoxin, for instance a maytansinoid, a dolastatin, an auristatin drug analogue, a cryptophycin, duocarmycin, a duocarmycin deriative, an enediyne antibiotic, or a pyrolobenodiazepine. In some embodiments, the payload is a genetically active nucleic acid (i.e. a nucleic acid that interacts with the translational and/or transcriptional machinery). Thus, in some embodiments, the nucleic acid does not act as an adjuvant. In these embodiments, the nucleic acid is not a TLR agonist. More particularly, in these embodiments, the nucleic acid is not Poly I:C (polyinosine-polycytidylic acid), which binds TLR3; is not polyU RNA (1-(2-methylpropyl)-1H-imidazo(4,5-c)quinolin-4-amine), which binds TLR7; and is not CpG (DNA CpG motifs), which binds TLR9. Instead, the nucleic acid may encode a gene product, or it may encode (or be) an interfering RNA.

In embodiments where the nucleic acid encodes a gene product, the gene product may be an antigen, e.g. a tumour antigen or a viral antigen, and the binding agent/payload complex can be used as a vaccine.

### Sequences

In view of the present disclosure, the amino acid sequences of the domains of the following receptors can be spliced together to form chimeric receptors of the invention:

### Human DNGR-1 (CLEC9A)

Human DNGR-1 is a type II transmembrane protein. The full length amino acid sequence of human DNGR-1 (CLEC9A) is available on the public protein databases, e.g. on the NCBI database with identifier NP_997228.1, and is provided here by SEQ ID NO:1:

(SEQ ID NO: 1)
MHEEEIYTSLQWDSPAPDTYQKCLSSNKCSGACCLVMVIS
CVFCMGLLTASIFLGVKLLQVSTIAMQQQEKLIQQERALL
NFTEWKRSCALQMKYCQAFMQNSLSSAHNSSPCPNNWIQN
RESCYYVSEIWSIWHTSQENCLKEGSTLLQIESKEEMDFI
TGSLRKIKGSYDYWVGLSQDGHSGRWLWQDGSSPSPGLLP
AERSQSANQVCGYVKSNSLLSSNCSTWKYFICEKYALRSS
V

(Some splice variants of human DNGR-1 exist, however they do not vary with respect to the cytoplasmic domain.)

### The Cytoplasmic Domain of Human DNGR-1

The part of the amino acid sequence of human DNGR-1 that is the N-terminal cytoplasmic domain is set forth in SEQ ID NO:2:

(SEQ ID NO: 2)
MHEEEIYTSLQWDSPAPDTYQKCLSSNKCSGA

### The Transmembrane Domain of Human DNGR-1

The part of the amino acid sequence of human DNGR-1 that is the transmembrane domain is set forth in SEQ ID NO:3:

(SEQ ID NO: 3)
CCLVMVISCVFCMGLLTASIFLGV

### The Neck Domain of Human DNGR-1

The part of the amino acid sequence of human DNGR-1 that is the neck domain is set forth in SEQ ID NO:4:

(SEQ ID NO: 4)
KLLQVSTIAMQQQEKLIQQERALLNFTEWKRSCALQMKYC
QAFMQNSLSSAHNSS

### The C-type Lectin Domain (CTLD) of Human DNGR-1

The part of the amino acid sequence of human DNGR-1 that is the CTLD is set forth in SEQ ID NO:5:

(SEQ ID NO: 5)
PCPNNWIQNRESCYYVSEIWSIWHTSQENCLKEGSTLLQIESKEEMDFIT
GSLRKIKGSYDYWVGLSQDGHSGRWLWQDGSSPSPGLLPAERSQSANQVC
GYVKSNSLLSSNCSTWKYFICEKYA

### Mouse DNGR-1 (CLEC9A)

Mouse DNGR-1 is a type II transmembrane protein. The full length amino acid sequence of mouse DNGR-1 (CLEC9A) is available on the public protein databases, e.g. on the NCBI database with identifier NP_001192292.1, and is provided here by SEQ ID NO:6:

(SEQ ID NO: 6)
MHAEEIYTSLOWDIPTSEASQKCQSPSKCSGAWCVVTMISCVVCMGLLAT
SIFLGIKFFQVSSLVLEQQERLIQQDTALVNLTQWQRKYTLEYCQALLQR
SLHSGTDASTGPVLLTSPQMVPQTLDSKETGSDCSPCPHNWIQNGKSCYY
VFERWEMWNISKKSCLKEGASLFQIDSKEEMEFISSIGKLKGGNKYWVGV
FQDGISGSWFWEDGSSPLSDLLPAERQRSAGQICGYLKDSTLISDKCDSW
KYFICEKKAFGSCI

### The Cytoplasmic Domain of Mouse DNGR-1

The part of the amino acid sequence of mouse DNGR-1 that is the N-terminal cytoplasmic domain is set forth in SEQ ID NO:7:

(SEQ ID NO: 7)
MHAEEIYTSLQWDIPTSEASQKCQSPSKCSGA

### The Transmembrane Domain of Mouse DNGR-1

The part of the amino acid sequence of mouse DNGR-1 that is the transmembrane domain is set forth in SEQ ID NO:8:

(SEQ ID NO: 8)
WCVVTMISCVVCMGLLATSIFLGI

### The Neck Domain of Mouse DNGR-1

The part of the amino acid sequence of mouse DNGR-1 that is the neck domain is set forth in SEQ ID NO:9:

(SEQ ID NO: 9)
KFFQVSSLVLEQQERLIQQDTALVNLTQWQRKYTLEYCQALLQRSLHSGT
DASTGPVLLTSPQMVPQTLDSKETGSDCS

### The C-Type Lectin Domain (CTLD) of Mouse DNGR-1

The part of the amino acid sequence of mouse DNGR-1 that is the CTLD is set forth in SEQ ID NO:10:

(SEQ ID NO: 10)
PCPHNWIQNGKSCYYVFERWEMWNISKKSCLKEGASLFQIDSKEEMEFIS
SIGKLKGGNKYWVGVFQDGISGSWFWEDGSSPLSDLLPAERQRSAGQICG
YLKDSTLISDKCDSWKYFICEKKA

### The Signalling Domain of the Cytoplasmic Tail of DNGR-1

The present disclosure shows that the cytoplasmic tail of DNGR-1 is a key mediator of XP and that its capacity to mediate XP requires only the DNGR-1 signalling domain. This sequence comprises a “HemITAM”⁵⁴ sequence that allows/mediates Syk binding when fused to mouse Dectin-1.

The signalling portion of the DNGR-1 cytoplasmic domain subsists in the first thirteen residues of the cytoplasmic domain, as set forth in SEQ ID NO:11:

MHAEEIYTSLQWD (SEQ ID NO:11). This sequence is the first thirteen residues of mouse DNGR-1.

The signalling portion of human DNGR-1 cytoplasmic domain subsists in the first thirteen residues of the cytoplasmic domain, as set forth in SEQ ID NO:89:

(SEQ ID NO: 89)
MHEEEIYTSLOWD

hemITAM Domains

The above 13-amino acid portion of the DNGR-1 cytoplasmic domain comprises a hemITAM domain, as set forth in SEQ ID NO:12:

(SEQ ID NO: 12)
EEIYTSL

Other hemITAM sequences have been reported (Baur and Steinle, 2017, which is hereby incorporated by reference in its entirety) such as that set forth in SEQ ID NO:13

(SEQ ID NO: 13)
DEDGYXXL

The essential amino acid residues of the hemITAM sequence are the first glutamic acid (E), the central tyrosine (Y) and the leucine (L) because these residues are conserved in hemITAM sequences across different proteins and in different species. Thus, the signalling domain of the chimeric receptor of the invention may comprise a hemITAM as set forth in SEQ ID NO:14:

EXXYXXL (SEQ ID NO:14), where ‘X’ represents any amino acid residue.

### Intracellular Signalling Domains

The inventors believe that the 1-3 amino acids on each side of the hemITAM of DNGR-1 may be important for promoting XP. Thus, the chimeric receptor of the invention may comprise an intracellular signalling domain (which may be denoted a “Syk-binding sequence derived from the signalling domain of the cytoplasmic tail of DNGR-1”) that comprises a sequence as set forth in SEQ ID NO:15:
MHAEXXYXXLQWD (SEQ ID NO:15), where ‘X’ represents any amino acid residue.

### Mouse Dectin-1 (CLEC7A)

Mouse Dectin-1 is a type II transmembrane protein. The full length amino acid sequence of mouse Dectin-1 (CLEC7A) is available on the public protein databases, e.g. on the NCBI database with identifier AAS37670.1, and is provided here by SEQ ID NO:16:

(SEQ ID NO: 16)
MKYHSHIENLDEDGYTQLDFSTQDIHKRPRGSEKGSRAPSSPWRPIAVGL
GILCFVVVVVAAVLGALAFWRHNSGRNPEEKDSFLSRNKENHKPTESSLD
EKVAPSKASQTTGGFSQSCLPNWIMHGKSCYLFSFSGNSWYGSKRHCSQL
GAHLLKIDNSKEFEFIESQTSSHRINAFWIGLSRNQSEGPWFWEDGSAFF
PNSFQVRNAVPQESLLHNCVWIHGSEVYNQICNTSSYSICEKEL

### The Cytoplasmic Domain of Mouse Dectin-1

The part of the amino acid sequence of mouse Dectin-1 that is the N-terminal cytoplasmic domain is set forth in SEQ ID NO:17:

(SEQ ID NO: 17)
MKYHSHIENLDEDGYTQLDFSTQDIHKRPRGSEKGSRAPSSPWR

### The Transmembrane Domain of Mouse Dectin-1

The part of the amino acid sequence of mouse Dectin-1 that is the transmembrane domain is set forth in SEQ ID NO:18:

(SEQ ID NO: 18)
VGLGILCFVVVVVAAVLGALAFW

### The Neck Domain of Mouse Dectin-1

The part of the amino acid sequence of mouse Dectin-1 that is the neck domain is set forth in SEQ ID NO:19:

(SEQ ID NO: 19)
RHNSGRNPEEKDSFLSRNKENHKPTESSLDEKVAPSKASQTTGGFSQSCL
PNWIM

### The C-Type Lectin Domain (CTLD) of Mouse Dectin-1

The part of the amino acid sequence of mouse Dectin-1 that is the CTLD is set forth in SEQ ID NO:20:

(SEQ ID NO: 20)
HGKSCYLFSFSGNSWYGSKRHCSQLGAHLLKIDNSKEFEFIESQTSSHRI
NAFWIGLSRNQSEGPWFWEDGSAFFPNSFQVRNAVPQESLLHNCVWIHGS
EVYNQICNTSSYSICE

### Human FcR Gamma Chain

Human FcR gamma chain is type I transmembrane protein. The full length amino acid sequence of human FcR gamma chain is available on the public protein databases, e.g. on the NCBI database with identifier NP_004097, and is provided here by SEQ ID NO:21:

(SEQ ID NO: 21)
MIPAVVLLLLLLVEQAAALGEPQLCYILDAILFLYGIVLTLLYCRLKIQV
RKAAITSYEKSDGVYTGLSTRNQETYETLKHEKPPQ

### The Cytoplasmic Domain of Human FcR Gamma Chain

The part of the amino acid sequence of human FcR gamma chain that is the C-terminal cytoplasmic domain is set forth in SEQ ID NO:22:

(SEQ ID NO: 22)
RLKIQVRKAAITSYEKSDGVYTGLSTRNQETYETLKHEKPPQ

### The Transmembrane Domain of Human FcR Gamma Chain

The part of the amino acid sequence of human FcR gamma chain that is the transmembrane domain is set forth in SEQ ID NO:23:

(SEQ ID NO: 23)
LCYILDAILFLYGIVLTLLYC

### The Extracellular Domain of Human FcR Gamma Chain

The part of the amino acid sequence of human FcR gamma chain that is the extracellular domain is set forth in SEQ ID NO:24:

(SEQ ID NO: 24)
LGEPQ

### Mouse FcR Gamma Chain

Mouse FcR gamma chain is type I transmembrane protein. The full length amino acid sequence of mouse FcR gamma chain is available on the public protein databases, e.g. on the NCBI database with identifier NP_034315.1, and is provided here by SEQ ID NO:25:

(SEQ ID NO: 25)
MISAVILFLLLLVEQAAALGEPQLCYILDAVLFLYGIVLTLLYCRLKIQV
RKAAIASREKADAVYTGLNTRSQETYETLKHEKPPQ

### The Cytoplasmic Domain of Mouse FcR Gamma Chain

The part of the amino acid sequence of mouse FcR gamma chain that is the C-terminal cytoplasmic domain is set forth in SEQ ID NO:26:

(SEQ ID NO: 26)
RLKIQVRKAAIASREKADAVYTGLNTRSQETYETLKHEKPPQ

### The Transmembrane Domain of Mouse FcR Gamma Chain

The part of the amino acid sequence of mouse FcR gamma chain that is the transmembrane domain is set forth in SEQ ID NO:27:

(SEQ ID NO: 27)
LCYILDAVLFLYGIVLTLLYC

### The Extracellular Domain of Mouse FcR Gamma Chain

The part of the amino acid sequence of mouse FcR gamma chain that is the extracellular domain is set forth in SEQ ID NO:28:

(SEQ ID NO: 28)
LGEPQ

### Human FcγRI

Human FcγRI is a type I transmembrane protein. The full length amino acid sequence of human FcγRI is available on the public protein databases, e.g. on the NCBI database with identifier NP_001365733, and is provided here by SEQ ID NO:29:

(SEQ ID NO: 29)
MWFLTTLLLWVPVDGQVDTTKAVITLQPPWVSVFQEETVTLHCEVLHLPG
SSSTQWFLNGTATQTSTPSYRITSASVNDSGEYRCQRGLSGRSDPIQLEI
HRGWLLLQVSSRVFTEGEPLALRCHAWKDKLVYNVLYYRNGKAFKFFHWN
SNLTILKTNISHNGTYHCSGMGKHRYTSAGISVTVKELFPAPVLNASVTS
PLLEGNLVTLSCETKLLLQRPGLQLYFSFYMGSKTLRGRNTSSEYQILTA
RREDSGLYWCEAATEDGNVLKRSPELELQVLGLQLPTPVWFHVLFYLAVG
IMFLVNTVLWVTIRKELKRKKKWDLEISLDSGHEKKVISSLQEDRHLEEE
LKCQEQKEEQLQEGVHRKEPQGAT

### The Cytoplasmic Domain of Human FcγRI

The part of the amino acid sequence of human FcγRI that is the C-terminal cytoplasmic domain is set forth in SEQ ID NO:30:

(SEQ ID NO: 30)
RKELKRKKKWDLEISLDSGHEKKVISSLQEDRHLEEELKCQEQKEEQLQE
GVHRKEPQGAT

### The Transmembrane Domain of Human FcγRI

The part of the amino acid sequence of human FcγRI that is the transmembrane domain is set forth in SEQ ID NO:31:

(SEQ ID NO: 31)
VLFYLAVGIMFLVNTVLWVTI

### The Extracellular Domain of Human FcγRI

The part of the amino acid sequence of human FcγRI that is the extracellular domain is set forth in SEQ ID NO:32:

(SEQ ID NO: 32)
QVDTTKAVITLQPPWVSVFQEETVTLHCEVLHLPGSSSTQWFLNGTATQT
STPSYRITSASVNDSGEYRCQRGLSGRSDPIQLEIHRGWLLLQVSSRVFT
EGEPLALRCHAWKDKLVYNVLYYRNGKAFKFFHWNSNLTILKTNISHNGT
YHCSGMGKHRYTSAGISVTVKELFPAPVLNASVTSPLLEGNLVTLSCETK
LLLQRPGLQLYFSFYMGSKTLRGRNTSSEYQILTARREDSGLYWCEAATE
DGNVLKRSPELELQVLGLQLPTPVWFH

### Mouse FcγRI

Mouse FcγRI is a type I transmembrane protein. The full length amino acid sequence of mouse FcγRI is available on the public protein databases, e.g. on the NCBI database with identifier NP_034316, and is provided here by SEQ ID NO:33:

(SEQ ID NO: 33)
MILTSFGDDMWLLTTLLLWVPVGGEVVNATKAVITLQPPWVSIFQKENVT
LWCEGPHLPGDSSTQWFINGTAVQISTPSYSIPEASFQDSGEYRCQIGSS
MPSDPVQLQIHNDWLLLQASRRVLTEGEPLALRCHGWKNKLVYNVVFYRN
GKSFQFSSDSEVAILKTNLSHSGIYHCSGTGRHRYTSAGVSITVKELFTT
PVLRASVSSPFPEGSLVTLNCETNLLLQRPGLQLHFSFYVGSKILEYRNT
SSEYHIARAEREDAGFYWCEVATEDSSVLKRSPELELQVLGPQSSAPVWF
HILFYLSVGIMFSLNTVLYVKIHRLQREKKYNLEVPLVSEQGKKANSFQQ
VRSDGVYEEVTATASQTTPKEAPDGPRSSVGDCGPEQPEPLPPSDSTGAQ
TSQS

### The Cytoplasmic Domain of Mouse FcγRI

The part of the amino acid sequence of mouse FcγRI that is the C-terminal cytoplasmic domain is set forth in SEQ ID NO:34:

(SEQ ID NO: 34)
KIHRLQREKKYNLEVPLVSEQGKKANSFQQVRSDGVYEEVTATASQTTPK
EAPDGPRSSVGDCGPEQPEPLPPSDSTGAQTSQS

### The Transmembrane Domain of Mouse FcγRI

The part of the amino acid sequence of mouse FcγRI that is the transmembrane domain is set forth in SEQ ID NO:35:

(SEQ ID NO: 35)
VWFHILFYLSVGIMFSLNTVLYV

### The Extracellular Domain of Mouse FcγRI

The part of the amino acid sequence of mouse FcγRI that is the extracellular domain is set forth in SEQ ID NO:36:

(SEQ ID NO: 36)
EVVNATKAVITLQPPWVSIFQKENVTLWCEGPHLPGDSSTQWFINGTAVQ
ISTPSYSIPEASFQDSGEYRCQIGSSMPSDPVQLQIHNDWLLLQASRRVL
TEGEPLALRCHGWKNKLVYNVVFYRNGKSFQFSSDSEVAILKTNLSHSGI
YHCSGTGRHRYTSAGVSITVKELFTTPVLRASVSSPFPEGSLVTLNCETN
LLLQRPGLQLHFSFYVGSKILEYRNTSSEYHIARAEREDAGFYWCEVATE
DSSVLKRSPELELQVLGPQSSAP

### Human FcγRIIA

Human FcγRIIA is a type I transmembrane protein. The full length amino acid sequence of human FcγRIIA is available on the public protein databases, e.g. on the NCBI database with identifier NP_001129691, and is provided here by SEQ ID NO:37:

(SEQ ID NO: 37)
MTMETQMSQNVCPRNLWLLQPLTVLLLLASADSQAAAPPKAVLKLEPPWI
NVLQEDSVTLTCQGARSPESDSIQWFHNGNLIPTHTQPSYRFKANNNDSG
EYTCQTGQTSLSDPVHLTVLSEWLVLQTPHLEFQEGETIMLRCHSWKDKP
LVKVTFFQNGKSQKFSHLDPTFSIPQANHSHSGDYHCTGNIGYTLFSSKP
VTITVQVPSMGSSSPMGIIVAVVIATAVAAIVAAVVALIYCRKKRISANS
TDPVKAAQFEPPGRQMIAIRKRQLEETNNDYETADGGYMTLNPRAPTDDD
KNIYLTLPPNDHVNSNN

### The Cytoplasmic Domain of Human FcγRIIA

The part of the amino acid sequence of human FcγRIIA that is the C-terminal cytoplasmic domain is set forth in SEQ ID NO:38:

(SEQ ID NO: 38)
CRKKRISANSTDPVKAAQFEPPGRQMIAIRKRQLEETNNDYETADGGYMT
LNPRAPTDDDKNIYLTLPPNDHVNSNN

### The Transmembrane Domain of Human FcγRIIA

The part of the amino acid sequence of human FcγRIIA that is the transmembrane domain is set forth in SEQ ID NO:39:

(SEQ ID NO: 39)
IIVAVVIATAVAAIVAAVVALIY

### The Extracellular Domain of Human FcγRIIA

The part of the amino acid sequence of human FcγRIIA that is the extracellular domain is set forth in SEQ ID NO:40:

(SEQ ID NO: 40)
QAAAPPKAVLKLEPPWINVLQEDSVTLTCQGARSPESDSIQWFHNGNLIP
THTQPSYRFKANNNDSGEYTCQTGQTSLSDPVHLTVLSEWLVLQTPHLEF
QEGETIMLRCHSWKDKPLVKVTFFQNGKSQKFSHLDPTFSIPQANHSHSG
DYHCTGNIGYTLFSSKPVTITVQVPSMGSSSPMG

### Human TIMD4

Human TIMD4 is a type I transmembrane protein. The full length amino acid sequence of human TIMD4 is available on the public protein databases, e.g. on the NCBI database with identifier NP_612388, and is provided here by SEQ ID NO:41:

(SEQ ID NO: 41)
MSKEPLILWLMIEFWWLYLTPVTSETVVTEVLGHRVTLPCLYSSWSHNSN
SMCWGKDQCPYSGCKEALIRTDGMRVTSRKSAKYRLQGTIPRGDVSLTIL
NPSESDSGVYCCRIEVPGWFNDVKINVRLNLQRASTTTHRTATTTTRRTT
TTSPTTTRQMTTTPAALPTTVVTTPDLTTGTPLQMTTIAVFTTANTCLSL
TPSTLPEEATGLLTPEPSKEGPILTAESETVLPSDSWSSVESTSADTVLL
TSKESKVWDLPSTSHVSMWKTSDSVSSPQPGASDTAVPEQNKTTKTGQMD
GIPMSMKNEMPISQLLMIIAPSLGFVLFALFVAFLLRGKLMETYCSQKHT
RLDYIGDSKNVLNDVQHGREDEDGLFTL

### The Cytoplasmic Domain of Human TIMD4

The part of the amino acid sequence of human TIMD4 that is the C-terminal cytoplasmic domain is set forth in SEQ ID NO:42:

(SEQ ID NO: 42)
LRGKLMETYCSQKHTRLDYIGDSKNVLNDVQHGREDEDGLFTL

### The Transmembrane Domain of Human TIMD4

The part of the amino acid sequence of human TIMD4 that is the transmembrane domain is set forth in SEQ ID NO:43:

(SEQ ID NO: 43)
LLMIIAPSLGFVLFALFVAFL

### The Extracellular Domain of Human TIMD4

The part of the amino acid sequence of human TIMD4 that is the extracellular domain is set forth in SEQ ID NO:44:

(SEQ ID NO: 44)
ETVVTEVLGHRVTLPCLYSSWSHNSNSMCWGKDQCPYSGCKEALIRTDGM
RVTSRKSAKYRLQGTIPRGDVSLTILNPSESDSGVYCCRIEVPGWFNDVK
INVRLNLQRASTTTHRTATTTTRRTTTTSPTTTRQMTTTPAALPTTVVTT
PDLTTGTPLQMTTIAVFTTANTCLSLTPSTLPEEATGLLTPEPSKEGPIL
TAESETVLPSDSWSSVESTSADTVLLTSKESKVWDLPSTSHVSMWKTSDS
VSSPQPGASDTAVPEQNKTTKTGQMDGIPMSMKNEMPISQ

### Mouse TIMD4

Mouse TIMD4 is a type I transmembrane protein. The full length amino acid sequence of mouse TIMD4 is available on the public protein databases, e.g. on the NCBI database with identifier NP_848874, and is provided here by SEQ ID NO:45:

(SEQ ID NO: 45)
MSKGLLLLWLVTELWWLYLTPAASEDTIIGFLGQPVTLPCHYLSWSQSRN
SMCWGKGSCPNSKCNAELLRTDGTRIISRKSTKYTLLGKVQFGEVSLTIS
NTNRGDSGVYCCRIEVPGWFNDVKKNVRLELRRATTTKKPTTTTRPTTTP
YVTTTTPELLPTTVMTTSVLPTTTPPQTLATTAFSTAVTTCPSTTPGSFS
QETTKGSAFTTESETLPASNHSQRSMMTISTDIAVLRPTGSNPGILPSTS
QLTTQKTTLTTSESLQKTTKSHQINSRQTILIIACCVGFVLMVLLFLAFL
LRGKVTGANCLQRHKRPDNTEDSDSVLNDMSHGRDDEDGIFTL

### The Cytoplasmic Domain of Mouse TIMD4

The part of the amino acid sequence of mouse TIMD4 that is the C-terminal cytoplasmic domain is set forth in SEQ ID NO:46:

(SEQ ID NO: 46)
LRGKVTGANCLQRHKRPDNTEDSDSVLNDMSHGRDDEDGIFTL

### The Transmembrane Domain of Mouse TIMD4

The part of the amino acid sequence of

mouse TIMD4 that is the transmembrane domain is set forth in SEQ ID NO:88:

(SEQ ID NO: 88)
ILIIACCVGFVLMVLLFLAFL

### The Extracellular Domain of Mouse TIMD4

The part of the amino acid sequence of mouse TIMD4 that is the extracellular domain is set forth in SEQ ID NO:47:

(SEQ ID NO: 47)
ASEDTIIGFLGQPVTLPCHYLSWSQSRNSMCWGKGSCPNSKCNAELLRTD
GTRIISRKSTKYTLLGKVQFGEVSLTISNTNRGDSGVYCCRIEVPGWFND
VKKNVRLELRRATTTKKPTTTTRPTTTPYVTTTTPELLPTTVMTTSVLPT
TTPPQTLATTAFSTAVTTCPSTTPGSFSQETTKGSAFTTESETLPASNHS
QRSMMTISTDIAVLRPTGSNPGILPSTSQLTTQKTTLTTSESLQKTTKSH
QINSRQT

### Human Megf10

Human Megf10 is a type I transmembrane protein. The full length amino acid sequence of human Megf10 is available on the public protein databases, e.g. on the NCBI database with identifier NP_001243474, and is provided here by SEQ ID NO:48:

(SEQ ID NO: 48)
MVISLNSCLSFICLLLCHWIGTASPLNLEDPNVCSHWESYSVTVQESYPH
PFDQIYYTSCTDILNWFKCTRHRVSYRTAYRHGEKTMYRRKSQCCPGFYE
SGEMCVPHCADKCVHGRCIAPNTCQCEPGWGGTNCSSACDGDHWGPHCTS
RCQCKNGALCNPITGACHCAAGFRGWRCEDRCEQGTYGNDCHQRCQCQNG
ATCDHVTGECRCPPGYTGAFCEDLCPPGKHGPQCEQRCPCQNGGVCHHVT
GECSCPSGWMGTVCGQPCPEGRFGKNCSQECQCHNGGTCDAATGQCHCSP
GYTGERCQDECPVGTYGVLCAETCQCVNGGKCYHVSGACLCEAGFAGERC
EARLCPEGLYGIKCDKRCPCHLENTHSCHPMSGECACKPGWSGLYCNETC
SPGFYGEACQQICSCQNGADCDSVTGKCTCAPGFKGIDCSTPCPLGTYGI
NCSSRCGCKNDAVCSPVDGSCTCKAGWHGVDCSIRCPSGTWGFGCNLTCQ
CLNGGACNTLDGTCTCAPGWRGEKCELPCQDGTYGLNCAERCDCSHADGC
HPTTGHCRCLPGWSGVHCDSVCAEGRWGPNCSLPCYCKNGASCSPDDGIC
ECAPGFRGTTCQRICSPGFYGHRCSQTCPQCVHSSGPCHHITGLCDCLPG
FTGALCNEVCPSGRFGKNCAGICTCTNNGTCNPIDRSCQCYPGWIGSDCS
QPCPPAHWGPNCIHTCNCHNGAFCSAYDGECKCTPGWTGLYCTQRCPLGF
YGKDCALICQCQNGADCDHISGQCTCRTGFMGRHCEQKCPSGTYGYGCRQ
ICDCLNNSTCDHITGTCYCSPGWKGARCDQAGVIIVGNLNSLSRTSTALP
ADSYQIGAIAGIIILVLVVLFLLALFIIYRHKQKGKESSMPAVTYTPAMR
VVNADYTISGTLPHSNGGNANSHYFTNPSYHTLTQCATSPHVNNRDRMTV
TKSKNNQLFVNLKNVNPGKRGPVGDCTGTLPADWKHGGYLNELGAFGLDR
SYMGKSLKDLGKNSEYNSSNCSLSSSENPYATIKDPPVLIPKSSECGYVE
MKSPARRDSPYAEINNSTSANRNVYEVEPTVSVVQGVFSNNGRLSQDPYD
LPKNSHIPCHYDLLPVRDSSSSPKQEDSGGSSSNSSSSSE

### The Cytoplasmic Domain of Human Megf10

The part of the amino acid sequence of human Megf10_that is the C-terminal cytoplasmic domain is set forth in SEQ ID NO:49:

(SEQ ID NO: 49)
YRHKQKGKESSMPAVTYTPAMRVVNADYTISGTLPHSNGGNANSHYFTNP
SYHTLTQCATSPHVNNRDRMTVTKSKNNQLFVNLKNVNPGKRGPVGDCTG
TLPADWKHGGYLNELGAFGLDRSYMGKSLKDLGKNSEYNSSNCSLSSSEN
PYATIKDPPVLIPKSSECGYVEMKSPARRDSPYAEINNSTSANRNVYEVE
PTVSVVQGVFSNNGRLSQDPYDLPKNSHIPCHYDLLPVRDSSSSPKQEDS
GGSSSNSSSSSE

### The Transmembrane Domain of Human Megf10

The part of the amino acid sequence of human Megf10 that is the transmembrane domain is set forth in SEQ ID NO:50:

(SEQ ID NO: 50)
AIAGIIILVLVVLFLLALFII

### The Extracellular Domain of Human Megf10

The part of the amino acid sequence of human Megf10 that is the extracellular domain is set forth in SEQ ID NO:51:

(SEQ ID NO: 51)
LNLEDPNVCSHWESYSVTVQESYPHPFDQIYYTSCTDILNWFKCTRHRVS
YRTAYRHGEKTMYRRKSQCCPGFYESGEMCVPHCADKCVHGRCIAPNTCQ
CEPGWGGTNCSSACDGDHWGPHCTSRCQCKNGALCNPITGACHCAAGFRG
WRCEDRCEQGTYGNDCHQRCQCQNGATCDHVTGECRCPPGYTGAFCEDLC
PPGKHGPQCEQRCPCQNGGVCHHVTGECSCPSGWMGTVCGQPCPEGRFGK
NCSQECQCHNGGTCDAATGQCHCSPGYTGERCQDECPVGTYGVLCAETCQ
CVNGGKCYHVSGACLCEAGFAGERCEARLCPEGLYGIKCDKRCPCHLENT
HSCHPMSGECACKPGWSGLYCNETCSPGFYGEACQQICSCQNGADCDSVT
GKCTCAPGFKGIDCSTPCPLGTYGINCSSRCGCKNDAVCSPVDGSCTCKA
GWHGVDCSIRCPSGTWGFGCNLTCQCLNGGACNTLDGTCTCAPGWRGEKC
ELPCQDGTYGLNCAERCDCSHADGCHPTTGHCRCLPGWSGVHCDSVCAEG
RWGPNCSLPCYCKNGASCSPDDGICECAPGFRGTTCQRICSPGFYGHRCS
QTCPQCVHSSGPCHHITGLCDCLPGFTGALCNEVCPSGRFGKNCAGICTC
TNNGTCNPIDRSCQCYPGWIGSDCSQPCPPAHWGPNCIHTCNCHNGAFCS
AYDGECKCTPGWTGLYCTQRCPLGFYGKDCALICQCQNGADCDHISGQCT
CRTGFMGRHCEQKCPSGTYGYGCRQICDCLNNSTCDHITGTCYCSPGWKG
ARCDQAGVIIVGNLNSLSRTSTALPADSYQIG

### Mouse Megf10

Mouse Megf10 is a type I transmembrane protein. The full length amino acid sequence of mouse Megf10 is available on the public protein databases, e.g. on the NCBI database with identifier NP_001001979, and is provided here by SEQ ID NO:52:

(SEQ ID NO: 52)
MAISSSSCLGLICSLLCHWVGTASSLNLEDPNVCSHWESYSVTVQESYPH
PFDQIYYTSCTDILNWFKCTRHRISYRTAYRHGEKTMYRRKSQCCPGFYE
SRDMCVPHCADKCVHGRCIAPNTCQCEPGWGGTNCSSACDGDHWGPHCSS
RCQCKNRALCNPITGACHCAAGYRGWRCEDRCEQGTYGNDCHQRCQCQNG
ATCDHITGECRCSPGYTGAFCEDLCPPGKHGPHCEQRCPCQNGGVCHHVT
GECSCPSGWMGTVCGQPCPEGRFGKNCSQECQCHNGGTCDAATGQCHCSP
GYTGERCQDECPVGSYGVRCAEACRCVNGGKCYHVSGTCLCEAGFSGELC
EARLCPEGLYGIKCDKRCPCHLDNTHSCHPMSGECGCKPGWSGLYCNETC
SPGFYGEACQQICSCQNGADCDSVTGRCACAPGFKGTDCSTPCPLGRYGI
NCSSRCGCKNDAVCSPVDGSCICKAGWHGVDCSIRCPSGTWGFGCNLTCQ
CLNGGACNTLDGTCTCAPGWRGAKCEFPCQDGTYGLNCAERCDCSHADGC
HPTTGHCRCLPGWSGVHCDSVCAEGRWGPNCSLPCYCKNGASCSPDDGIC
ECAPGFRGTTCQRICSPGFYGHRCSQTCPQCVHSSGPCHHITGLCDCLPG
FTGALCNEVCPSGRFGKNCAGVCTCTNNGTCNPIDRSCQCYPGWIGSDCS
QPCPPAHWGPNCIHTCNCHNGAFCSAYDGECKCTPGWTGLYCTQRCPLGF
YGKDCALICQCQNGADCDHISGQCTCRTGFMGRHCEQKCPAGTYGYGCRQ
ICDCLNNSTCDHITGTCYCSPGWKGARCDQAGVIIVGNLNSLSRTSTALP
ADSYQIGAIAGIVVLVLVVLFLLALFIIYRHKQKRKESSMPAVTYTPAMR
VINADYTIAETLPHSNGGNANSHYFTNPSYHTLSQCATSPHVNNRDRMTI
AKSKNNQLFVNLKNVNPGKRGTLVDCTGTLPADWKQGGYLNELGAFGLDR
SYMGKSLKDLGKNSEYNSSTCSLSSSENPYATIKDPPALLPKSSECGYVE
MKSPARRDSPYAEINNSTPANRNVYEVEPTVSVVQGVFSNSGHVTQDPYD
LPKNSHIPCHYDLLPVRDSSSSPKREDGGGSNSTSSNSTSSSSSSSE

### The Cytoplasmic Domain of Mouse Megf10

The part of the amino acid sequence of mouse Megf10 that is the C-terminal cytoplasmic domain is set forth in SEQ ID NO:53:

(SEQ ID NO: 53)
YRHKQKRKESSMPAVTYTPAMRVINADYTIAETLPHSNGGNANSHYFTNP
SYHTLSQCATSPHVNNRDRMTIAKSKNNQLFVNLKNVNPGKRGTLVDCTG
TLPADWKQGGYLNELGAFGLDRSYMGKSLKDLGKNSEYNSSTCSLSSSEN
PYATIKDPPALLPKSSECGYVEMKSPARRDSPYAEINNSTPANRNVYEVE
PTVSVVQGVFSNSGHVTQDPYDLPKNSHIPCHYDLLPVRDSSSSPKREDG
GGSNSTSSNSTSSSSSSSE

### The Transmembrane Domain of Mouse Megf10

The part of the amino acid sequence of mouse Megf10 that is the transmembrane domain is set forth in SEQ ID NO:54:

(SEQ ID NO: 54)
AIAGIVVLVLVVLFLLALFII

### The Extracellular Domain of Mouse Megf10

The part of the amino acid sequence of mouse Megf10 that is the extracellular domain is set forth in SEQ ID NO:55:

(SEQ ID NO: 55)
LNLEDPNVCSHWESYSVTVQESYPHPFDQIYYTSCTDILNWFKCTRHRIS
YRTAYRHGEKTMYRRKSQCCPGFYESRDMCVPHCADKCVHGRCIAPNTCQ
CEPGWGGTNCSSACDGDHWGPHCSSRCQCKNRALCNPITGACHCAAGYRG
WRCEDRCEQGTYGNDCHQRCQCQNGATCDHITGECRCSPGYTGAFCEDLC
PPGKHGPHCEQRCPCQNGGVCHHVTGECSCPSGWMGTVCGQPCPEGRFGK
NCSQECQCHNGGTCDAATGQCHCSPGYTGERCQDECPVGSYGVRCAEACR
CVNGGKCYHVSGTCLCEAGFSGELCEARLCPEGLYGIKCDKRCPCHLDNT
HSCHPMSGECGCKPGWSGLYCNETCSPGFYGEACQQICSCQNGADCDSVT
GRCACAPGFKGTDCSTPCPLGRYGINCSSRCGCKNDAVCSPVDGSCICKA
GWHGVDCSIRCPSGTWGFGCNLTCQCLNGGACNTLDGTCTCAPGWRGAKC
EFPCQDGTYGLNCAERCDCSHADGCHPTTGHCRCLPGWSGVHCDSVCAEG
RWGPNCSLPCYCKNGASCSPDDGICECAPGFRGTTCQRICSPGFYGHRCS
QTCPQCVHSSGPCHHITGLCDCLPGFTGALCNEVCPSGRFGKNCAGVCTC
TNNGTCNPIDRSCQCYPGWIGSDCSQPCPPAHWGPNCIHTCNCHNGAFCS
AYDGECKCTPGWTGLYCTQRCPLGFYGKDCALICQCQNGADCDHISGQCT
CRTGFMGRHCEQKCPAGTYGYGCRQICDCLNNSTCDHITGTCYCSPGWKG
ARCDQAGVIIVGNLNSLSRTSTALPADSYQIG

### Human CD3 Zeta Chain

Human CD3 zeta chain is a type I transmembrane protein. The full length amino acid sequence of human CD3 zeta chain is available on the public protein databases, e.g. on the NCBI database with identifier NP_932170, and is provided here by SEQ ID NO:56:

(SEQ ID NO: 56)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

### The Cytoplasmic Domain of Human CD3 Zeta Chain

The part of the amino acid sequence of human CD3 zeta chain_that is the C-terminal cytoplasmic domain is set forth in SEQ ID NO:57:

(SEQ ID NO: 57)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR

### The Transmembrane Domain of Human CD3 Zeta Chain

The part of the amino acid sequence of human CD3 zeta chain_that is the transmembrane domain is set forth in SEQ ID NO:58:

(SEQ ID NO: 58)
LCYLLDGILFIYGVILTALFL

### The Extracellular Domain of Human CD3 Zeta Chain

The part of the amino acid sequence of human CD3 zeta chain_that is the extracellular domain is set forth in SEQ ID NO:59:

(SEQ ID NO: 59)
QSFGLLDPK

### Mouse CD3 Zeta Chain

Mouse CD3 zeta chain is a type I transmembrane protein. The full length amino acid sequence of mouse CD3 zeta chain is available on the public protein databases, e.g. on the NCBI database with identifier NP_001106862, and is provided here by SEQ ID NO:60:

(SEQ ID NO: 60)
MKWKVSVLACILHVRFPGAEAQSFGLLDPKLCYLLDGILFIYGVIITALY
LRAKFSRSAETAANLQDPNQLYNELNLGRREEYDVLEKKRARDPEMGGKQ
QRRRNPQEGVYNALQKDKMAEAYSEIGTKGERRRGKGHDGLYQGLSTATK
DTYDALHMQTLAPR

### The Cytoplasmic Domain of Mouse CD3 Zeta Chain

The part of the amino acid sequence of mouse CD3 zeta chain that is the C-terminal cytoplasmic domain is set forth in SEQ ID NO:61:

(SEQ ID NO: 61)
RAKFSRSAETAANLQDPNQLYNELNLGRREEYDVLEKKRARDPEMGGKQQ
RRRNPQEGVYNALQKDKMAEAYSEIGTKGERRRGKGHDGLYQGLSTATKD
TYDALHMQTLAPR

### The Transmembrane Domain of Mouse CD3 Zeta Chain

The part of the amino acid sequence of mouse CD3 zeta chain that is the transmembrane domain is set forth in SEQ ID NO:62:

(SEQ ID NO: 62)
LCYLLDGILFIYGVIITALYL

### The Extracellular Domain of Mouse CD3 Zeta Chain

The part of the amino acid sequence of mouse CD3 zeta chain that is the extracellular domain is set forth in SEQ ID NO:63:

(SEQ ID NO: 63)
QSFGLLDPK

### Human Transferrin Receptor

The human transferrin receptor is a type II transmembrane protein. The full length amino acid sequence of the human transferrin receptor is available on the public protein databases, e.g. on the NCBI database with identifier NP_001121620, and is provided here by SEQ ID NO:64:

(SEQ ID NO: 64)
MMDQARSAFSNLFGGEPLSYTRFSLARQVDGDNSHVEMKLAVDEEENADN
NTKANVTKPKRCSGSICYGTIAVIVFFLIGFMIGYLGYCKGVEPKTECER
LAGTESPVREEPGEDFPAARRLYWDDLKRKLSEKLDSTDFTGTIKLLNEN
SYVPREAGSQKDENLALYVENQFREFKLSKVWRDQHFVKIQVKDSAQNSV
IIVDKNGRLVYLVENPGGYVAYSKAATVTGKLVHANFGTKKDFEDLYTPV
NGSIVIVRAGKITFAEKVANAESLNAIGVLIYMDQTKFPIVNAELSFFGH
AHLGTGDPYTPGFPSFNHTQFPPSRSSGLPNIPVQTISRAAAEKLFGNME
GDCPSDWKTDSTCRMVTSESKNVKLTVSNVLKEIKILNIFGVIKGFVEPD
HYVVVGAQRDAWGPGAAKSGVGTALLLKLAQMFSDMVLKDGFQPSRSIIF
ASWSAGDFGSVGATEWLEGYLSSLHLKAFTYINLDKAVLGTSNFKVSASP
LLYTLIEKTMQNVKHPVTGQFLYQDSNWASKVEKLTLDNAAFPFLAYSGI
PAVSFCFCEDTDYPYLGTTMDTYKELIERIPELNKVARAAAEVAGQFVIK
LTHDVELNLDYERYNSQLLSFVRDLNQYRADIKEMGLSLQWLYSARGDFF
RATSRLTTDFGNAEKTDRFVMKKLNDRVMRVEYHFLSPYVSPKESPFRHV
FWGSGSHTLPALLENLKLRKQNNGAFNETLFRNQLALATWTIQGAANALS
GDVWDIDNEF

### The Cytoplasmic Domain of Human Transferrin Receptor

The part of the amino acid sequence of human transferrin receptor that is the C-terminal cytoplasmic domain is set forth in SEQ ID NO:65:

(SEQ ID NO: 65)
MMDQARSAFSNLFGGEPLSYTRFSLARQVDGDNSHVEMKLAVDEEENADN
NTKANVTKPKRCSGSIC

### The Transmembrane Domain of Human Transferrin Receptor

The part of the amino acid sequence of human transferrin receptor that is the transmembrane domain is set forth in SEQ ID NO:66:

(SEQ ID NO: 66)
YGTIAVIVFFLIGFMIGYLGY

### The Extracellular Domain of Human Transferrin Receptor

The part of the amino acid sequence of human transferrin receptor that is the extracellular domain is set forth in SEQ ID NO:67:

(SEQ ID NO: 67)
CKGVEPKTECERLAGTESPVREEPGEDFPAARRLYWDDLK
RKLSEKLDSTDFTGTIKLLNENSYVPREAGSQKDENLALY
VENQFREFKLSKVWRDQHFVKIQVKDSAQNSVIIVDKNGR
LVYLVENPGGYVAYSKAATVTGKLVHANFGTKKDFEDLYT
PVNGSIVIVRAGKITFAEKVANAESLNAIGVLIYMDQTKF
PIVNAELSFFGHAHLGTGDPYTPGFPSFNHTQFPPSRSSG
LPNIPVQTISRAAAEKLFGNMEGDCPSDWKTDSTCRMVTS
ESKNVKLTVSNVLKEIKILNIFGVIKGFVEPDHYVVVGAQ
RDAWGPGAAKSGVGTALLLKLAQMFSDMVLKDGFQPSRSI
IFASWSAGDFGSVGATEWLEGYLSSLHLKAFTYINLDKAV
LGTSNFKVSASPLLYTLIEKTMQNVKHPVTGQFLYQDSNW
ASKVEKLTLDNAAFPFLAYSGIPAVSFCFCEDTDYPYLGT
TMDTYKELIERIPELNKVARAAAEVAGQFVIKLTHDVELN
LDYERYNSQLLSFVRDLNQYRADIKEMGLSLOWLYSARGD
FFRATSRLTTDFGNAEKTDRFVMKKLNDRVMRVEYHFLSP
YVSPKESPFRHVFWGSGSHTLPALLENLKLRKQNNGAFNE
TLFRNQLALATWTIQGAANALSGDVWDIDNEF

### The Extracellular Domain of Human Transferrin Receptor

The part of the amino acid sequence of human transferrin receptor that is the ligand binding domain is set forth in SEQ ID NO:68:

(SEQ ID NO: 68)
EDTDYPYLGTTMDTYKELIERIPELNKVARAAAEVAGQFV
IKLTHDVELNLDYERYNSQLLSFVRDLNQYRADIKEMGLS
LOWLYSARGDFFRATSRLTTDFGNAEKTDRFVMKKLNDRV
MRVEYHFLSPYVSPKESPFRHVFWGSGSHTLPALLENLKL
RKQNNGAFNETLFRNQLALATWTIQGAANALSGDVWDIDN
EF

### Human Clec-2 Receptor

The human Clec-2 receptor is a type II transmembrane protein. The full length amino acid sequence of the human Clec-2 receptor is available on the public protein databases, e.g. on the NCBI database with identifier NP_057593 and is provided here by SEQ ID NO:69:

(SEQ ID NO: 69)
MQDEDGYITLNIKTRKPALISVGSASSSWWRVMALILLIL
CVGMVVGLVALGIWSVMQRNYLQGENENRTGTLQQLAKRF
CQYVVKQSELKGTFKGHKCSPCDTNWRYYGDSCYGFFRHN
LTWEESKQYCTDMNATLLKIDNRNIVEYIKARTHLIRWVG
LSRQKSNEVWKWEDGSVISENMFEFLEDGKGNMNCAYFHN
GKMHPTFCENKHYLMCERKAGMTKVDQLP

### The Cytoplasmic Domain of Human Clec-2 Receptor

The part of the amino acid sequence of human Clec-2 receptor that is the C-terminal cytoplasmic domain is set forth in SEQ ID NO:70:

(SEQ ID NO: 70)
MQDEDGYITLNIKTRKPALISVGSASSSWWRVM

### The Transmembrane Domain of Human Clec-2 Receptor

The part of the amino acid sequence of human Clec-2 receptor that is the transmembrane domain is set forth in SEQ ID NO:71:

(SEQ ID NO: 71)
ALILLILCVGMVVGLVALGIW

### The Extracellular Domain of Human Clec-2 Receptor

The part of the amino acid sequence of human Clec-2 receptor that is the extracellular domain is set forth in SEQ ID NO:72:

(SEQ ID NO: 72)
SVMQRNYLQGENENRTGTLQQLAKRFCQYVVKQSELKGTF
KGHKCSPCDTNWRYYGDSCYGFFRHNLTWEESKQYCTDMN
ATLLKIDNRNIVEYIKARTHLIRWVGLSRQKSNEVWKWED
GSVISENMFEFLEDGKGNMNCAYFHNGKMHPTFCENKHYL
MCERKAGMTKVDQLP

### Human KLRF1 Receptor

The human KLRF1 receptor is a type II transmembrane protein. The full length amino acid sequence of the human KLRF1 receptor is available on the public protein databases, e.g. on the NCBI database with identifier Q9NZS2 and is provided here by SEQ ID NO:73:

(SEQ ID NO: 73)
MQDEERYMTLNVQSKKRSSAQTSQLTFKDYSVTLHWYKIL
LGISGTVNGILTLTLISLILLVSQGVLLKCQKGSCSNATQ
YEDTGDLKVNNGTRRNISNKDLCASRSADQTVLCQSEWLK
YQGKCYWFSNEMKSWSDSYVYCLERKSHLLIIHDQLEMAF
IQKNLRQLNYVWIGLNFTSLKMTWTWVDGSPIDSKIFFIK
GPAKENSCAAIKESKIFSETCSSVFKWICQY

### The Cytoplasmic Domain of Human KLRF1 Receptor

The part of the amino acid sequence of human KLRF1 receptor that is the C-terminal cytoplasmic domain is set forth in SEQ ID NO:74:

(SEQ ID NO: 74)
MQDEERYMTLNVQSKKRSSAQTSQLTFKDYSVTLHWYK

### The Transmembrane Domain of Human KLRF1 Receptor

The part of the amino acid sequence of human KLRF1 receptor that is the transmembrane domain is set forth in SEQ ID NO:75:

(SEQ ID NO: 75)
ILLGISGTVNGILTLTLISLI

### The Extracellular Domain of Human KLRF1 Receptor

The part of the amino acid sequence of human KLRF1 receptor that is the extracellular domain is set forth in SEQ ID NO:76:

(SEQ ID NO: 76)
LLVSQGVLLKCQKGSCSNATQYEDTGDLKVNNGTRRNISN
KDLCASRSADQTVLCQSEWLKYQGKCYWFSNEMKSWSDSY
VYCLERKSHLLIIHDQLEMAFIQKNLRQLNYVWIGLNFTS
LKMTWTWVDGSPIDSKIFFIKGPAKENSCAAIKESKIFSE
TCSSVFKWICQY

### Human Dectin-1 Receptor

The human Dectin-1 receptor is a type II transmembrane protein. The full length amino acid sequence of the human Dectin-1 receptor is available on the public protein databases, e.g. on the NCBI database with identifier NP_922938 and is provided here by SEQ ID NO:77:

(SEQ ID NO: 77)
MEYHPDLENLDEDGYTQLHFDSQSNTRIAVVSEKGSCAAS
PPWRLIAVILGILCLVILVIAVVLGTMAIWRSNSGSNTLE
NGYFLSRNKENHSQPTQSSLEDSVTPTKAVKTTGVLSSPC
PPNWIIYEKSCYLFSMSLNSWDGSKRQCWQLGSNLLKIDS
SNELGFIVKQVSSQPDNSFWIGLSRPQTEVPWLWEDGSTF
SSNLFQIRTTATQENPSPNCVWIHVSVIYDQLCSVPSYSI
CEKKFSM

### The Cytoplasmic Domain of Human Dectin-1 Receptor

The part of the amino acid sequence of human Dectin-1 receptor that is the C-terminal cytoplasmic domain is set forth in SEQ ID NO:78:

(SEQ ID NO: 78)
MEYHPDLENLDEDGYTQLHFDSQSNTRIAVVSEKGSCAASPPWR

### The Transmembrane Domain of Human Dectin-1 Receptor

The part of the amino acid sequence of human Dectin-1 receptor that is the transmembrane domain is set forth in SEQ ID NO:79:

(SEQ ID NO: 79)
LIAVILGILCLVILVIAVVLG

### The Extracellular Domain of Human Dectin-1 Receptor

The part of the amino acid sequence of human Dectin-1 receptor that is the extracellular domain is set forth in SEQ ID NO:80:

(SEQ ID NO: 80)
TMAIWRSNSGSNTLENGYFLSRNKENHSQPTQSSLEDSVT
PTKAVKTTGVLSSPCPPNWIIYEKSCYLFSMSLNSWDGSK
RQCWQLGSNLLKIDSSNELGFIVKQVSSQPDNSFWIGLSR
PQTEVPWLWEDGSTFSSNLFQIRTTATQENPSPNCVWIHV
SVIYDQLCSVPSYSICEKKFSM

The sequence information disclosed herein provides exemplary cytosolic, transmembrane and extracellular domains that can be combined to make further exemplary chimeric receptors of the invention. However, the chimeric receptors of the invention are defined by the claims and are not limited to the exemplary sequences disclosed above, unless specified as such in the claims.

### Chimeric Proteins

Chimeric proteins, such as those provided by the present invention, comprise sequences derived from more than one wildtype protein. For instance, the chimeric protein of the invention has an intracellular sequence that comprises an amino acid sequence derived from a DNGR-1 protein fused to sequences derived from other (non-DNGR-1) proteins. Thus the extracellular domain of the chimeric protein of the invention is functionally distinct from the extracellular domain of wild type DNGR-1 in some respects. For instance, the chimeric protein of the invention may not bind to the F-actin/myosin complexes, which are the natural ligands of wild type human DNGR-1 (and bind the CTLD at the extracellular domain of DNGR-1). For instance, anti-DNGR-1 antibodies, which specifically bind the extracellular domain of wild type human DNGR-1, may not specifically bind the chimeric proteins of the invention. Besides these functional differences, because the extracellular domain of the chimeric protein of the invention is not derived from DNGR-1, it has low sequence identity to the extracellular domain of DNGR-1. For instance, the extracellular domain of the chimeric protein of the invention may have less than 50% sequence identity to the extracellular domain of wild type human DNGR-1, as measured across the length of the DNGR-1 extracellular domain. In some embodiments, the extracellular domain of the chimeric protein of the invention has less than 40% sequence identity, less than 30% sequence identity, less than 25% sequence identity, or less than 20% sequence identity to the extracellular domain of wild type human DNGR-1, as measured across the length of the DNGR-1 extracellular domain.

### Sequence Relationships

It is routine within the fields of biochemistry and molecular biology to identify related sequences. The skilled person can readily determine whether a subject sequence is related to a reference sequence. If the sequences are related to each other, it could be said that one of the sequences is “derived from” the other.

Once the skilled person understands which amino acid residues are important to the function of a first protein, s/he can then determine whether a second sequence has these amino acid residues, appropriately positioned, and will therefore likely share the function of the first protein; if so, the sequences are structurally and functionally related. In the case of the amino acid sequences of the present invention, a “Syk-binding sequence derived from the signalling domain of the cytoplasmic tail of DNGR-1” is structurally and functionally related to the signalling domain of the cytoplasmic tail of DNGR-1 because it shares the key amino acid sequence motif of the cytoplasmic tail of DNGR-1 and it therefore shares the ability to bind Syk and facilitate XP via the cytosolic pathway. The tyrosine residue in the middle of the Syk-binding sequence represented by SEQ ID NO:15 (MHAEXXYXXLQWD) or SEQ ID NO:90 (MHEEXXYXXLQWD) is important for the Syk-binding function. In contrast, the methionine residue at position 1 is less likely to be important for function although it is needed for translation of constructs where the Syk-binding sequence sits at the N-terminus of the chimeric protein. It may be omitted from chimeric constructs in which an additional leader sequence or linker is present at the N-terminus. These constructs may therefore comprise SEQ ID NO:81 (HAEXXYXXLQWD) or SEQ ID NO:82 (HEEXXYXXLQWD). Additionally, the three amino acid residues at the C-terminal end of the Syk-binding sequence are not thought to be as important. Thus, the chimeric proteins of the invention may comprise SEQ ID NO:83 (MHAEXXYXXL), SEQ ID NO:84 (MHEEXXYXXL), SEQ ID NO:85 (HAEXXYXXL), or SEQ ID NO:86 (HEEXXYXXL). The chimeric protein of the invention may comprise SEQ ID NO:87 (HXEXXYXXL).

Additionally or alternatively, a degree of sequence identity can be specified to define the structural relationship between two sequences. For instance, a degree of sequence identity may be specified from at least 60% to 100% sequence identity. More preferably, the specified degree of sequence identity may be one of at least 65%, 70%, 75%, 80%, 85%, 86%, 87%, 88%, 89%, 90%, 91%, 92%, 93%, 94%, 95%, 96%, 97%, 98% or 99% identity.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1. Phagosomal damage. mCherry-galectin-3 was expressed in DNGR-1 deficient MuTuDCs reconstituted with either WT or mutant (Y7F) DNGR-1 and cells were incubated with FM-OVA beads. Galectin-3 phagosome⁺ cells were counted and plotted as a ratio (index) to bead+ cells. Galectin-3 binds sugar moieties attached to membrane proteins on the luminal side of damaged endosomes and phagosomes. mCherry-galectin-3 was recruited to phagosomes in cells expressing wildtype (WT) but not the tyrosine-to-phenylalanine (Y7F) mutant.

FIG. 2. Efficient XP in non-professional APCs expressing a chimeric receptor of the invention, ‘C9/C7’ (shown as “C9::C7”). IL-2 ELISA from B3Z hybridoma and HEK293T C7, C9/C7 or C9(Y7F)/C7 (shown as “C9(Y7F)::C7”) cell co-culture supernatants stimulated with zymosan-OVA. Data plotted as mean±standard deviation of an experimental triplicate.

FIG. 3. Syk phosphorylation. Quantification of fold enrichment of phospho-SYK-staining on phagosomes (n>50 phagosomes) in HEK293T C7, C7(Y15F), C9/C7 (shown as “C9::C7”) and C9(Y7F)/C7 (shown as “C9(Y7F)::C7”) cells. p values were calculated by two-tailed Mann-Whitney test. Representative plots (n=2).

### DETAILED DESCRIPTION OF THE INVENTION

This disclosure shows that the superiority of cDC1s in XP is not fully dependent on unique cell biology but also on the expression of receptors such as DNGR-1 that detect relevant XP substrates and initiate the intracellular signalling that allows phagosome disruption and enables efficient XP. The inventors show that ligand-dependent DNGR-1 signalling at the level of phagosomes induces a local NADPH-dependent oxidative burst that destabilises the phagosomal membrane causing rupture and wholesale access of luminal contents to the cytoplasmic compartment where they can enter the endogenous MHC class I processing pathway. Notably, the ability of DNGR-1 to signal for phagosomal rupture is intrinsic to its cytoplasmic signalling domain and can be transplanted onto other receptors and other cell types. Thus, XP relies on ubiquitous machinery for reactive oxygen species production in endosomes that can be subverted by specialised receptors to deliberately provoke vacuolar membrane damage and P2C.

These results do not exclude the fact that cDC1 possess cell biological specialisations that favour Xp^{7,30,36,40-42}. Indeed, we identify in these cells slowly maturing phagosomal compartments that can retain undegraded cargo for long periods, which is known to favour XP^{22,23}. The fact that DNGR-1 preferentially localises to these early phagosomes but does not impact their maturation is consistent with the notion that the main function of the receptor is to survey vacuolar compartments for the presence of exposed F-actin/myosin complexes, indicative of putatively antigenic cargo that is relatively intact. Receptor engagement then leads to Syk-dependent local production of ROS and membrane damage. Rupture of any given phagosome is likely to be a stochastic event partly determined by the extent of damage possibly offset by membrane repair. Phagosomes that do not rupture can continue to mature, generating the LAMP⁺ DNGR-1⁻ degradative late phagosome pool that we also detect in our assays. The limited nature of the rupture event, and the fact that it is circumscribed to early non-degradative endosomes, may contribute to preventing cell toxicity that would be expected to be induced by introduction of proteolytic enzymes into the cytosol⁴⁹. Yet, the probability of rupture is sufficiently high that all C9/C7-expressing HEK293T cells die upon overnight incubation with cytochrome c-soaked zymosan, which indicates at least one phagosome rupture event in each of the cells within the 24 h period. Other receptors that can signal via Syk (e.g., integrins) might also plug into the phagosomal damage pathway with varying efficiency, which could explain instances of XP with ligands such as latex beads that have been shown to engage a Vav-Rac-NADPH oxidase-dependent XP pathway³⁵. However, some receptors that can target antigens for XP by cDC1, such as the mannose receptor, may employ a distinct mechanism of P2C^{44,50}. Furthermore, it is clear that not all Syk-activating receptors cause phagosomal damage and P2C, arguing for signal divergence at the level of Syk activation. Most notably, Dectin-1, the canonical Syk-coupled hemITAM-bearing receptor⁵¹, does not induce XP but, rather, promotes DC activation and inflammatory gene expression⁵². Conversely, DNGR-1 signaling triggers XP but does not induce DC activation⁴, which means that DNGR-1 acts exclusively as a receptor to decode the antigenicity of internalised cargo. Thus, additional signals emanating from dead cells are required to activate cross-presenting cDC1 and render them competent to prime CD8⁺ T cells (e.g., for anti-tumour immunity), as previously noted in the context of antibody-mediated antigen targeting to DNGR-1 where adjuvants are necessary for inducing a productive CTL response¹¹. Because activation signals can also impact XP⁵³, further understanding of how they synergise with signals emanating from dedicated XP-promoting receptors such as DNGR-1 offers great promise for the design of immunotherapies and vaccines that harness the power of CD8⁺ T cells.

### Professional/Non-Professional Antigen Presenting Cells

Some immune cells such as dendritic cells and macrophages are considered to be “professional” antigen presenting cells (professional APCs) because they are specialised to perform this function. While many cell types can perform some degree of antigen presentation upon MHC class I molecules (under certain conditions, specifically when the antigen has been synthesised intracellularly), professional APCs can present antigens upon MHC class II molecules in addition to presenting antigens on MHC class I. By expressing DNGR-1 or the chimeric receptor of the invention, cells that are not professional APCs, can be enabled to cross-present antigens on MHC I. For instance, by expressing DNGR-1 or the chimeric receptor of the invention in fibroblasts or muscle cells (which are non-professional APCs), these cells can be enabled to cross-present antigens on MHC I.

### Biopolymers

Biopolymers are polymeric biomolecules, which are produced in nature in biological systems such as cells and which may also be produced by biotechnological systems, such as cell free expression systems. Biopolymers include polypeptides, polynucleotides and polysaccharides. Molecules that comprise a polypeptide, polynucleotide and/or polysaccharide domain are considered to be biopolymers even if the peptide, nucleotide, or saccharide sequence is not found in nature and/or if the molecule comprises additional non-biomolecule and/or non-polymer domains.

### Pharmaceutical Compositions

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204(s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the vectors, cells and or chimeric receptors, used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

### Subject

The subject to be treated may be any animal or human. The subject is preferably mammalian, more preferably human. The subject may be a non-human mammal, but is more preferably human. The subject may be male or female. The subject may be a patient. Therapeutic uses may be in human or animals (veterinary use).

### Cancers

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chrondrosarcoma, osteosarcoma).

Some cancers cause solid tumours. Such solid tumours may be located in any tissue, for example the pancreas, lung, breast, uterus, stomach, kidney or testis. In contrast, cancers of the blood, such as leukaemias, may not cause solid tumours—and may be referred to as liquid tumours.

### Vectors

A “vector” as used herein is an oligonucleotide molecule (DNA or RNA) used as a vehicle to transfer foreign genetic material into a cell. The vector may be an expression vector for expression of the foreign genetic material in the cell. Such vectors may include a promoter sequence operably linked to the nucleotide sequence encoding the gene sequence to be expressed. A vector may also include a termination codon and expression enhancers. Any suitable vectors, promoters, enhancers and termination codons known in the art may be used to express the chimeric receptor of the invention in a cell or tissue.

The skilled person will appreciate that a gene therapy vector can be used to introduce the nucleic acid of the invention into a recipient cell or tissue. In some embodiments, the gene therapy vector is a viral vector. The viral vector may be an adenoviral vector, an AAV or a lentiviral vector. For some applications, it is advantageous to use a viral vector that is pseudotyped with an envelope protein that facilitates the transduction of hematopoietic stem cells and/or progenitor cells. In some embodiments, the nucleic acid is introduced into the mammalian cell using the CRISPR-CAS9 system.

### Antibody-Based Target Binding Domains

Antibodies which will bind to the targets discussed herein are already known. In view of today's techniques in relation to monoclonal antibody technology, antibodies can be prepared to most antigens.

The target binding domain may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799). Monoclonal antibodies (mAbs) are useful in the methods of the invention and are a homogenous population of antibodies specifically targeting a single epitope on an antigen. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al (1988, 8th International Biotechnology Symposium Part 2, 792-799)).

Polyclonal antibodies are useful in the methods of the invention. Monospecific polyclonal antibodies are preferred. Suitable polyclonal antibodies can be prepared using methods well known in the art. Fragments of antibodies, such as Fab and Fab₂ fragments may also be used as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. directly, by a peptide or by a flexible oligopeptide. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to a target discussed herein may also be made using phage display technology as is well known in the art (e.g. see “Phage display: a molecular tool for the generation of antibodies—a review”. Placenta. 21 Suppl A: S106-12. Helen E. Chadd and Steven M. Chamow; “Phage antibodies: filamentous phage displaying antibody variable domains”. Nature 348 (6301): 552-554).

Antibodies can be readily conjugated to peptide or protein moieties by operably linking a nucleotide sequence encoding the desired peptide or protein at the 3′ end of a nucleotide sequence that encodes one of the antibody chains, e.g. the heavy chain. Thus a fusion antibody can be expressed, where the C-terminus of the antibody polypeptide is fused to the desired peptide or protein, often via a linker sequence. Antibody fusions are well known in the art.

### Aptamer Based Target Binding Domains

Aptamers are short DNA/RNA/peptide molecules which can bind specifically to a target molecule (Pan & Clawson, 2009). Aptamers specific for a particular target are often selected from a large pool of randomly generated libraries of molecules, e.g. by using the Systematic Evolution of Ligands by Exponential Enrichment (SELEX) method. SELEX method involves exposing a random sequence library to a specific target and amplifying the bound molecules which are then subjected to additional rounds of selection. After multiple rounds of selection, specific aptamers identified for binding to the target molecule can be subjected to further rounds of modifications to improve their binding affinity and stability. Aptamers can be readily conjugated to additional nucleic acid moieties, thus facilitating targeted binding of the additional nucleic acid moiety to the specific binding target of the aptamer.

Aspects and embodiments of the present invention will now be discussed with reference to the accompanying figures. Further aspects and embodiments will be apparent to those skilled in the art. All documents mentioned in this text are incorporated herein by reference.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Example 1—DNGR-1 Knock-Out cDC1 Cells

To study the contribution of DNGR-1 to XP of dead cell-associated antigens by cDC1^{12,15}, the inventors made use of the cDC1 cell line MuTuDC1940 (henceforth termed MuTuDCs)¹⁷. MuTuDCs were pulsed with UV-irradiated ovalbumin (OVA)-expressing H-2K^{bm1} mouse embryonic fibroblasts (OVA dead cells) and then cultured with pre-activated OVA-specific (OT-I) CD8⁺ T cells. Interferon-γ (IFN-γ) accumulation in the culture indicates OT-I receptor triggering by H2K^{b}/OVA complexes and, hence, is an indirect measure of OVA XP by MuTuDCs. As reported¹⁸, OT-I T cultures with DNGR-1-deficient MuTuDCs (KO) accumulated lower levels of IFN-γ than cultures with wild type MuTuDCs (WT). This defect was corrected by ectopic re-expression of the receptor in DNGR-1-deficient MuTuDCs (KO-WT) and was not attributable to an effect on antigen uptake as KO, WT and KO-WT MuTuDCs all displayed a similar capacity to internalise dye-labelled dead cell material. In contrast, when studying the uptake of OVA-coated latex beads (OVA beads), we noticed that additional coating with the physiological ligand for DNGR-1, F-actin/myosin II complexes¹⁶ (FM-OVA beads), did result in greater bead internalisation (p<0.0001), which enhanced OVA XP (measured by IFN-γ production following co-culture with pre-activated OT-I CD8⁺ T cells, which enhanced OVA XP. Thus, DNGR-1 can serve as a phagocytic receptor for ligand-bearing particles but it is redundant for uptake of dead cell debris by cDC1, likely because the latter contain ligands that can engage additional cDC1 phagocytic receptors.

To separate the effect of DNGR-1 on XP from its contribution to ligand uptake, the inventors fed MuTuDCs with OVA beads or FM-OVA beads and sorted cells that had phagocytosed a single bead before testing them in the XP assay¹⁹. It was found that MuTuDCs containing single FM-OVA beads stimulated CD8⁺ OT-I T cells more efficiently than cells with single OVA beads. This effect was shown to be specific for XP, because both sets of sorted MuTuDCs (FM-OVA bead-uptake; and OVA bead-uptake) stimulated CD4⁺ OT-II T cells to the same extent.

DNGR-1-deficient MuTuDCs re-expressing either wildtype receptor (KO-WT), or a mutant receptor that cannot bind F-actin (W155A/W250A; termed KO-2WA) were compared, which showed a defect in XP of dead cell-associated antigen in KO-2WA cells but unaltered capacity to cross-present OVA beads or egg white (a source of endotoxin-free soluble OVA antigen). KO-WA MuTuDC internalised fewer FM-OVA beads than KO-WT cells and displayed markedly diminished ability to stimulate OT-I cells. Therefore, as above, taking single FM-OVA bead⁺ MuTuDCs, the inventors asked whether the mutation in the F-actin binding region of DNGR-1 impacted XP independently of particle uptake. Again, they observed a decrease in XP in cells bearing mutant DNGR-1 unable to engage its ligand. Together, these data indicate a specific effect of DNGR-1 engagement on XP of ligand-associated antigen, which can be formally distinguished from receptor contribution to ligand uptake.

### Example 2—The Cytosolic Pathway to Cross-Presentation

Two basic models for XP have emerged from studies in multiple cell types, one in which antigen processing and MHC class I molecule loading occurs entirely within the phago/endosomal compartment of APCs (“vacuolar” pathway) and another in which exogenous antigens somehow gain access to the APC cytosol (“cytosolic” or “phagosome-to-cytosol” (P2C) pathway) and are processed by the proteasome as for endogenous antigens²¹. Inhibition of lysosomal proteases by leupeptin or pepstatin, which block the vacuolar pathway, did not diminish XP of soluble antigen, bead-bound OVA or dead cell-associated OVA. In contrast, the proteasome inhibitor lactacystin inhibited XP of both bead-bound antigen and dead cell-associated antigen, but had no effect on XP of soluble antigen (except at high concentrations) or adverse effects on T cells stimulated with pre-processed OVA peptide SIINFEKL. In line with previous reports²²⁻²⁴, blockade of lysosomal acidification by chloroquine or blockade of cysteine proteases by E64 enhanced XP of FM-OVA beads and dead cell-associated antigen.

Taken together, these results suggest that antigen degradation through lysosomal proteases is detrimental for DNGR-1-dependent XP and that DNGR-1 engages the cytosolic rather than the vacuolar XP pathway in cDC1.

### Example 3—The DNGR-1⁺ Phagosomal Compartment

To investigate how DNGR-1 affects the properties of antigen-containing phagosomes, we isolated FM-OVA bead phagosomes from MuTuDCs and characterised them by flow cytometry (PhagoFACS). Interestingly, DNGR-1 and the lysosomal marker LAMP-2 marked two mutually exclusive phagosome populations, which, by microscopy were found to co-exist in individual cells. Further analysis revealed that DNGR-1⁺ phagosomes co-stained for MHC I and II whereas LAMP-2⁺ phagosomes were MHC II⁺ but contained lower levels of MHC I.

Importantly, the two phagosome populations showed differential capacity to degrade antigen, as DNGR-1⁺ phagosomes displayed high anti-OVA staining in both flow cytometry and microscopy assays whereas LAMP-2⁺ phagosomes stained only weakly. After long chase periods, some DNGR-1⁺ MHC I⁺ OVA^{high} phagosomes eventually lost DNGR-1 staining, acquired LAMP and degraded OVA, indicating that they were not fully arrested in maturation. DNGR-1⁺ MHC I⁺ OVA^{high} phagosomes were not an aberrant compartment of MuTu DCs as they were also found in phagosomal preparations derived from primary cDC1s obtained from bone marrow cultured in Flt3L and in KID cells, a second cDC1 cell line²⁶. Furthermore, ectopic expression of DNGR-1 in the macrophage cell line RAW264.7 also allowed for identification of DNGR-1⁺ OVA^{high} phagosomes distinct from LAMP-2⁺ OVA^{low} phagosomes. To ask if DNGR-1 was required for formation of MHC I⁺ OVA^{high} phagosomal compartments, the inventors compared FM-OVA bead phagosomes from DNGR-1 deficient (KO) and wildtype (WT) MuTuDCs. Interestingly, MHC I⁺ LAMP-2⁻ and MHC I⁺ OVA^{high} phagosomes were identified at similar frequency in both cells. We further compared DNGR-1⁺ phagosomes from wildtype MuTuDCs that had been fed OVA beads vs. FM-OVA beads and found no differences with respect to OVA degradation or MHC I recruitment.

Taken together, these data suggest that DNGR-1 marks an MHC I⁺ phagosomal compartment in cDC1 (and, when ectopically expressed, in RAW264.7 macrophages) that has low degradative potential and the ability to preserve undegraded antigen, at least temporarily. However, the presence of DNGR-1 or of its ligand does not affect the ability of phagocytic cargo to access this poorly degradative compartment.

### Example 4—Phagosome to Cytosol Transfer (P2C)

We searched for subsequent steps in XP that might be modulated by DNGR-1 ligand. As DNGR-1 dependent XP is mediated through the cytosolic pathway (see above), we focused on phagosome to cytosol transfer of the antigen. We investigated if DNGR-1⁺ phagosomes showed signs of membrane damage, consistent with disruption, by measuring local recruitment of cytosolic galectin-3 or 8, which bind to sugar moieties attached to membrane proteins on the luminal side of damaged endosomes and phagosomes²⁷. Strikingly, by PhagoFACS, we found that the frequency of galectin-8⁺ phagosomes in MuTuDCs was higher for DNGR-1⁺ phagosomes after 4 hours compared to LAMP-2⁺ phagosomes. Similarly, by confocal microscopy, mCherry-galectin-3 could be found decorating phagosomes when MuTuDCs were fed FM-OVA beads but much less frequently with OVA beads that do not trigger DNGR-1.

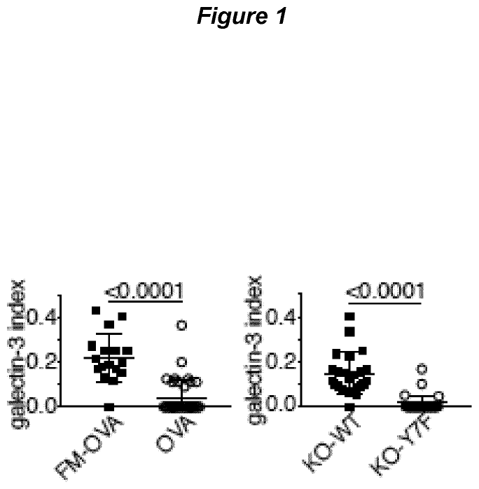

DNGR-1 is a type II trans-membrane protein with a short N-terminal intracellular tail bearing a single hemITAM signalling motif²⁸. To test the role of the DNGR-1 hemITAM, we expressed mCherry-galectin-3 in DNGR-1-deficient MuTuDCs reconstituted with either wildtype (KO-WT) or hemITAM signalling incompetent DNGR-1 (tyrosine to phenylalanine mutation at position 7—KO-Y7F). When cells were fed FM-OVA beads, mCherry-galectin-3 was recruited to phagosomes in cells expressing WT but not the Y7F receptor (FIG. 1).

To validate these observations, we used a different cytosolic sensor of endosomal membrane damage. Sphingomyelin is distributed asymmetrically in cellular membranes and is exposed to the cytosol only upon phagosomal damage. Therefore, cytosolic expression of a probe containing a version of the sphingomyelin-binding protein lysenin fused to mCherry allows for the specific labelling of damaged phagosomal membranes (Ellison et al, submitted). In line with the results with galectins, the mCherry-lysenin probe accumulated specifically on FM-OVA bead phagosomes when wildtype DNGR-1, but not Y7F DNGR-1, was expressed. The inventors conclude that ligand-dependent DNGR-1 signalling via its hemITAM induces phagosomal membrane damage in MutuDCs.

### Example 5—Chimeric Receptors Facilitate P2C

The inventors investigated the possibility that DNGR-1 hemITAM signalling could mediate phagosomal damage in a heterologous system. HEK293T cells were transfected with Dectin-1 (aka CLEC7A; a receptor structurally homologous to DNGR-1) or chimeras comprising the extracellular domain and transmembrane region of Dectin-1 fused to variants of the cytoplasmic tail of DNGR-1. Dectin-1 binds to yeast beta-glucans and functions as a yeast phagocytic receptor, allowing us to analyse uptake of zymosan particles (i.e., yeast cell walls) instead of latex beads. Indeed, Dectin-1 (C7), Dectin-1 fused with wildtype (C9/C7) or hemITAM tyrosine-mutated cytoplasmic tail of DNGR-1 (C9(Y7F)/C7) all conferred upon HEK293T the ability to take up zymosan while a tail-less mutant of Dectin-1 did not.

Notably, when co-expressing the mCherry-lysenin probe and the chimeric receptors in HEK293T, lysenin⁺ phagosomes were observed in cells expressing the C9/C7 chimera but not in cells expressing wild type C7 (p<0.0001) or the signaling incompetent chimera C9(Y7F)/C7 (p=0.03). In contrast to latex beads, zymosan particles are porous and do not have a solid core, therefore acting as a sponge for any probe that accesses the phagosomal lumen. Strikingly, we noticed that phagosomes in HEK293T cells expressing the C9/C7 chimera became positive for GFP that was expressed in the cytosol. In contrast, intra-phagosomal GFP was largely absent from zymosan-containing phagosomes in HEK293T expressing C7 or C9(Y7F)/C7, indicating a specific requirement for DNGR-1 hemITAM signalling.

Lysenin⁺ phagosomes showed a higher mean fluorescent intensity (MFI) for GFP compared to lysenin⁻ phagosomes, suggesting that access of cytosolic GFP to phagosomes was coupled to membrane damage. This was also apparent from live cell imaging, which showed that lysenin recruitment was predictive of but preceded GFP influx. These results suggest that DNGR-1 hemITAM signalling permeabilises phagosomes, rendering their lumen accessible to cytosolic proteins.

To assess whether permeability is bi-directional and permits release of phagosomal cargo into the cytosol, HEK293T expressing either C9/C7 or C9(Y7F)/C7 were pulsed with zymosan soaked in sulforhodamine B (SRB). A significant increase in SRB fluorescence was detected in the cytosol of HEK293T expressing C9/C7, but not those expressing C9(Y7F)/C7.

A previously-reported P2C assay²⁹ was also used to confirm that the efficacy with which zymosan-adsorbed beta-lactamase could be released from phagosomes into the cytosol was greater in HEK293T cells expressing C9/C7 than C9(Y7F)/C7.

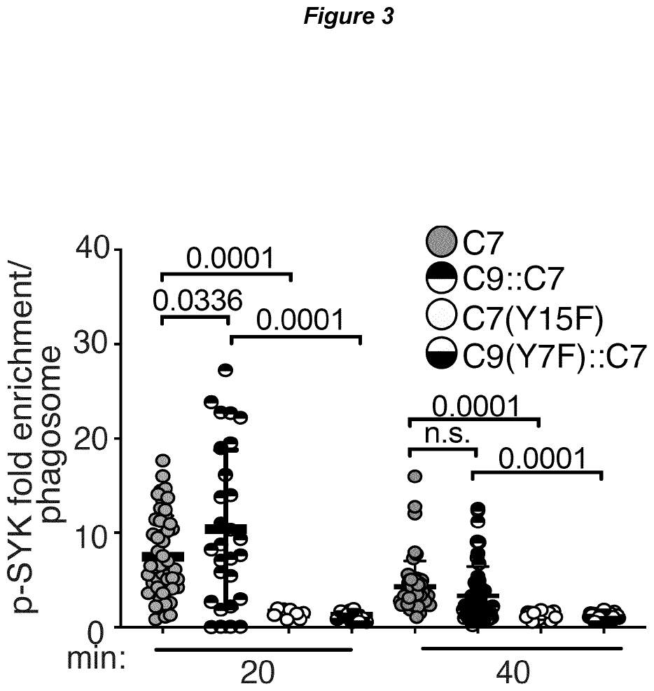

Zymosan soaked cytochrome c particles (zymosan-cyt. c particles) were added the to C7, C9/C7 and C9(Y7F)/C7 expressing HEK293T cells (FIG. 3g). When incubated with zymosan-cyt. c particles, HEK293T cells expressing C7 or C9(Y7F)/C7 internalised the particles but survival was unaffected. In stark contrast, all the C9/C7-expressing cells died within a 24 h period, demonstrating that DNGR-1 hemITAM-dependent P2C, essentially, occurs in all cells in which receptor signalling is triggered. Together, these results indicate that hemITAM signalling permeabilises phagosomes so as to allow efflux of luminal contents into the cytosol—and that this effect is retained when the hemITAM motif is present in the cytoplasmic tail of other transmembrane proteins, besides DNGR-1.

### Example 6—Characterising hemITAM-Induced Phagosome Permeability

To examine the nature and durability of DNGR-1 hemITAM-induced phagosomal permeability, the inventors performed FRAP experiments on GFP⁺ phagosomes stained with the lysenin probe. After photobleaching the GFP within the lumen of lysenin⁺ phagosomes, signal was re-observed within 2 minutes, indicating continuous GFP influx and suggesting irreversible phagosomal membrane damage. As a control, bleaching of the lysenin signal did not lead to fluorescence recovery.

Analysis of the ultrastructure of GFP⁺lysenin⁺ zymosan phagosomes in C9/C7-expressing cells by correlative light and electron microscopy revealed that the phagosomal membrane contained a large hole with a diameter of roughly 1-1.5 μm. Thus, DNGR-1 signalling can cause large scale rupture of phagosomes, which would allow for even sizeable luminal contents to be released into the cytosol.

### Example 7—XP in HEK293T Cells

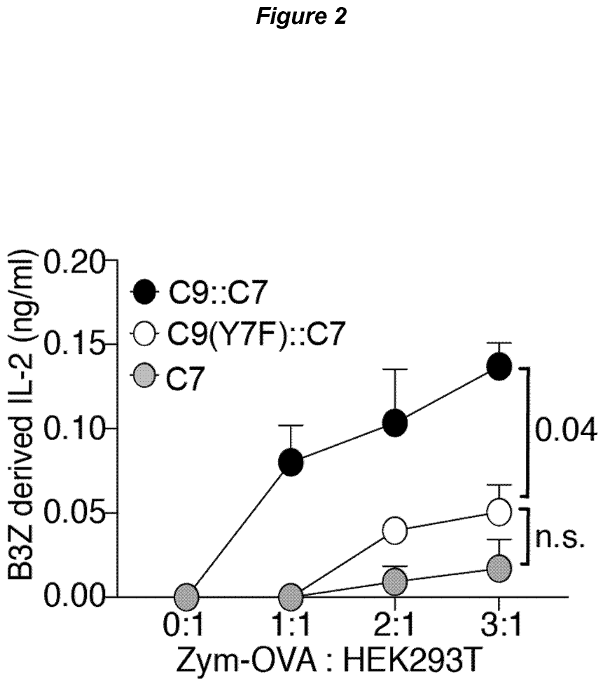

HEK293T lines stably expressing murine H-2K^{b} and beta-2-microglobulin were further transfected to express C7, C9/C7 or C9(Y7F)/C7 chimeras, single cell cloned and selected for equal H-2K^{b} and Dectin-1 extracellular domain expression levels. All three cell lines showed equivalent capacity to stimulate B3Z, an OVA/H-2K^{b}-specific T cell hybridoma, when pulsed with exogenous SIINFEKL peptide and were equally competent at presenting endogenous antigen when transfected to express Venus-SIINFEKL, a fusion protein mimic of endogenous OVA. These cells were exposed to zymosan that had been soaked in egg white (zymosan-OVA). C7, C9/C7and C9(Y7F)/C7 HEK293T lines all internalised zymosan-OVA with similar efficacy. However, efficient XP, as measured by B3Z activation after HEK293T cell fixation, was only observed with cells expressing the C9/C7 chimera (FIG. 2).

HEK293T cells expressing C9/C7 were then fed with zymosan dually soaked in both egg white and cytochrome c. Exposure time was optimised to kill only a fraction of the cells. This led to complete loss of cross presentation activity when compared to feeding cells with zymosan particles soaked with OVA alone. Overall cytotoxicity of cytochrome c in the culture was excluded by the fact that no decrease in B3Z activation was observed when we incubated zymosan-cyt. c particles with HEK293T cells expressing the C9/C7 chimera and Venus-SIINFEKL. These data formally indicate that DNGR-1 hemITAM signalling-induced phagosomal rupture is responsible for XP.

### Example 8—Interaction of the hemITAM Motif with the Tyrosine Kinase, Syk

The hemITAM motif of DNGR-1 can recruit and activate Syk or SHP-1 in response to DNGR-1 ligand engagement^{12,33}. Accordingly, Syk phosphorylation at two distinct sites was observed in wildtype MuTuDCs treated with anti-DNGR-1 cross-linking antibody or F-actin/myosin II complexes (DNGR-1 ligand; DNGR-1L). The inventors now confirm that C9/C7 chimeras also induce phosphorylation of Syk in HEK293T in response to zymosan treatment. This is observed at the level of phagosomes and is hemITAM-dependent, as it was not observed in cells expressing C9(Y7F)/C7 (FIG. 3).

To determine whether phosphorylation of Syk was upstream of phagosomal rupture, a Syk-deficient C9/C7-expressing HEK293T cell was generated using CRISPR/Cas9 (Syk^{CRISPR}) The influx of cytoplasmic GFP into zymosan phagosomes was completely lost in Syk^{CRISPR} cells. However, this influx was restored by reconstituting the Syk-deficient C9/C7-expressing HEK293T cells with (CRISPR-resistant) wild type mouse Syk (Syk WT) but not a catalytically-deficient mutant (K396R—kinase dead; Syk KD). Similarly, phagosomal GFP influx was not observed when C9/C7-expressing HEK293T cells were treated with the Syk inhibitor R406, even though zymosan uptake was not affected. In MuTu DCs, both R406 and another Syk inhibitor, inhibitor IV, abrogated staining of phagosomes with the lysenin probe and blocked XP of FM-OVA beads. This suggests that Syk activation and kinase activity downstream of DNGR-1 are required for the induction of phagosomal rupture and XP.

### Example 9—Syk Mediated Membrane Destabilisation and Rupture Via Reactive Oxygen Species

The inventors examined possible mechanisms downstream of Syk that might cause membrane destabilisation and rupture. Reactive oxygen species (ROS) produced by the NADPH oxidase cause lipid peroxidation leading to membrane destabilisation and endosomal content leakage³⁴⁻³⁷. Moreover, Syk activates NADPH oxidase activation via Vav and Rac, all of which have been implicated in XP of particulate antigens by myeloid cells³⁴⁻³⁷. The inventors noticed that exposure to zymosan led to a very potent oxidative burst in RAW264.7 cells ectopically expressing C9/C7 but not C9(Y7F)/C7. Using a fluorescent probe, the inventors confirmed that this burst occurred at the level of individual phagosomes and was diminished in RAW264.7 cells expressing C9(Y7F)/C7 or in cells treated with NADPH inhibitor, DPI. Similarly, in RAW264.7 cells expressing DNGR-1 (C9) and fed with fixed and permeabilised sheep red blood cells (a phagocytic target bearing exposed cortical F-actin that can be recognised by DNGR-1¹⁴), the inventors observed an oxidative burst around phagosomes that was diminished by SYK inhibition or DPI treatment. Similar results were obtained in HEK293T cells expressing C9/C7. Reactive oxygen species produced by NADPH cause lipid peroxidation and lead to membrane destabilisation and endosomal content leakage³⁷. Consistent with this notion, inhibition of NADPH oxidase by DPI prevented lysenin accumulation on phagosomes in RAW264.7 cells. Notably, it also decreased XP of zymosan-OVA by HEK293T cells whilst not affecting the presentation of Venus-SIINFEKL. Finally, in MuTu DCs, DPI, as well as the reactive oxygen species (ROS) scavenger, alpha-tocopherol (vitamin E) abrogated XP of FM-OVA beads or OVA-bearing dead cells but did not affect the presentation of SIINFEKL peptide. These results indicate that DNGR-1/Syk-dependent activation of NADPH causes lipid peroxidation allowing phagosomal rupture and P2C. This allows for the unselective release of internalised ligand-associated antigens into the cytosol of cDC1s and permits access to the endogenous MHC class I processing and presentation pathway.

Reactive oxygen species (ROS) produced by the NADPH oxidase can cause lipid peroxidation and lead to membrane destabilisation and endosomal content leakage³⁷. Consistent with a role for ROS in membrane damage, inhibition of the NADPH oxidase by DPI blocked lysenin accumulation on phagosomes in RAW264.7 cells expressing C9/C7 to the same level as the Y7F mutation. Furthermore, DPI, as well as the ROS scavenger alpha-tocopherol (vitamin E), greatly decreased XP of both FM-OVA beads and OVA-bearing dead cells in MutuDCs without impacting presentation of SIINFEKL peptide. Similarly, DPI decreased XP of zymosan-OVA by HEK293T cells expressing C9/C7 but did not diminish presentation of endogenous Venus-SIINFEKL antigen. Finally, siRNA-mediated silencing of NOX2 (CYBB), the predominant catalytic subunit of the NADPH oxidase in myeloid cells, decreased XP of zymosan-OVA by RAW264.7 expressing C9/C7 and H-2K^{b} but did not affect presentation of SIINFEKL peptide.

These data were extended to primary cDC1s by establishing Flt3L cultures with bone marrow from wild-type, DNGR-1 KO and NOX2 KO mice and purified cDC1s by magnetic enrichment. NOX2 KO cDC1 were defective in phagosomal ROS production in response to DNGR-1 stimulation and both DNGR-1 KO and NOX2 KO cDC1s displayed a reduction in XP of OVA-bearing dead cells relative to WT cDC1s despite being equally effective at presenting SIINFEKL peptide and internalising dead cell material. To assess the importance of NOX2 for DNGR-1-dependent XP in vivo, we first immunised wild-type, DNGR-1 KO, BATF3 KO or NOX2 KO mice with FM-OVA beads+poly I:C and measured OVA-specific CD8⁺ T cells responses by H-2K^{b}/OVA-pentamer staining. As reported¹⁶, WT mice mounted a robust response to FM-OVA beads+poly I:C that was significantly decreased in both DNGR-1 KO and BATF3 KO mice. Importantly, NOX2 KO mice also displayed a reduction in OVA-specific CD8⁺ T cell cross-priming. To confirm that this reflected NOX2 function in cDC1s and to extend the data to cross-priming to dead cell-associated antigens, we generated radiation chimeras using bone marrow from BATF3 KO CD45.1 mice mixed at a ratio of 80:20 with bone marrow from either BATF3 KO, wild-type, DNGR-1 KO or NOX2 KO CD45.2 mice. The inventors used BATF3 KO CD45.1 mice as recipients, further ensuring that the only cDC1 that develop after reconstitution arise from the CD45.2 donor bone marrow, and we analysed exclusively the response of CD45.1 T cells to exclude any cell-intrinsic effects of NOX2 deletion in lymphocytes. Following immunisation with OVA+polyl:C-pulsed dead cells, BATF3 KO:BATF3 KO chimeras failed to generate OVA-specific CD8⁺ T cells, as expected¹⁸. In contrast, robust cross-priming to OVA was seen in BATF3 KO:WT chimeras as measured by H-2K^{b}/OVA-tetramer staining or by intracellular staining for IFNγ in response to ex vivo spleen cell restimulation with SIINFEKL peptide. Consistent with previous observations in DNGR-1 KO mice^{3,11,12,44}, the OVA-specific CD8⁺ T cell response was significantly diminished in BATF3 KO:DNGR-1 KO chimeras. Notably, it was also significantly diminished in BATF3 KO:NOX2 KO chimeras.

Macrophages, monocyte-derived dendritic cells and other myeloid cell types, as well as non-immune cells, have been used extensively to dissect some of the mechanisms involved in XP^{7,23,45}. Relatively few papers have focused on XP mechanisms specifically in cDC1. Here, the inventors focus on the possibility that the superiority of cDC1s in XP depends not only on unique cell biology but also on the expression of receptors such as DNGR-1 that detect relevant XP substrates. The inventors show that ligand-dependent DNGR-1 signalling at the level of phagosomes induces a local NADPH-dependent oxidative burst that destabilises the phagosomal membrane causing rupture and wholesale access of luminal contents to the cytoplasmic compartment where they can enter the endogenous MHC I processing pathway. Notably, the ability of DNGR-1 to signal for phagosomal rupture is intrinsic to its cytoplasmic signalling domain and can be transplanted onto other receptors and operate in other cell types, including non-immune cells. Thus, XP relies on the machinery for reactive oxygen species production in endosomes. This machinery can be subverted by specialised receptors to deliberately provoke vacuolar membrane damage and P2C.

### NUMBERED PARAGRAPHS

1. A chimeric receptor comprising an extracellular target binding domain, a transmembrane domain, and an intracellular domain that comprises a Syk-binding sequence derived from the signalling domain of the cytoplasmic tail of DNGR-1, wherein said Syk-binding sequence contains a tyrosine residue.
2. The chimeric receptor according to paragraph 1, wherein the Syk-binding sequence comprises a hemITAM.
3. The chimeric receptor according to paragraph 2, wherein the hemITAM comprises the amino acid sequence set forth in SEQ ID NO:14 (EXXYXXL; wherein X represents any amino acid residue).
4. The chimeric receptor according to paragraph 3, wherein the Syk-binding sequence comprises an amino acid sequence as set forth in SEQ ID NO:15 (MHAEXXYXXLQWD) or as set forth in SEQ ID NO:90 (MHEEXXYXXLQWD); optionally wherein one, two or all three of the amino acid residues at the N-terminal end of the MHAEXXYXXLQWD (SEQ ID NO.: 15) or MHEEXXYXXLQWD (SEQ ID NO.: 90) sequence are removed or substituted with another amino acid residue, or wherein one, two or all three of the amino acid residues at the C-terminal end of the MHAEXXYXXLQWD (SEQ ID NO.: 15) sequence are removed or substituted with another amino acid residue.
5. The chimeric receptor according to any one of paragraphs 1-4, wherein the wherein the Syk-binding sequence comprises an amino acid sequence as set forth in SEQ ID NO:11 (MHAEEIYTSLQWD) or an amino acid sequence as set forth in SEQ ID NO:89 (MHEEEIYTSLQWD), optionally wherein one, two or three amino acid residues are substituted with another amino acid residue.
6. The chimeric receptor according to any one of the preceding paragraphs, wherein the target binding domain binds a target that is present on a pathogen, a pathogenic cell, a dead cell or a diseased cell.
7. The chimeric receptor according to paragraph 6, wherein the target is an antigen present on a tumour cell, optionally wherein the target is a tumour neoantigen.
8. The chimeric receptor according to paragraph 7, wherein the antigen present on the tumour cell is CEA, ERBB2, EGFR, GD2, mesothelin, MUC1, PSMA, CAIX, CD133, c-Met, EGFR, EGFRvIII, Epcam, EphA2, FRα, CD19, CD20, GPC3, GUCY2C, HER1, HER2, ICAM-1, MAGE, or MET.
9. The chimeric receptor according to paragraph 6, wherein the target is a viral antigen present at the surface of a viral particle or present on a virally infected cell.
10. The chimeric receptor according to paragraph 9, wherein the viral antigen is HCMV gB, influenza A hemagglutinin, influenza matrix 2 protein M2e, RSV glycoprotein F, SARS-Cov-2 Spike protein, HIV gp120 or HIV Env.
11. The chimeric receptor according to any one of the preceding paragraphs, wherein the target binding domain is derived from the ligand binding domain of a non-DNGR-1 lectin, a transferrin receptor, an FcR, an FcγRI, an FcγRIIA, TIMD4, Megf10, or a CD3 zeta chain.
12. The chimeric receptor according to paragraph 11, wherein the target binding domain is derived from mouse Dectin-1.
13. The chimeric receptor according to any one of the preceding paragraphs, wherein the target binding domain comprises an antibody variable region heavy chain (V_{H}) and/or light chain (V_{L}).
14. The chimeric receptor according to paragraph 13, wherein the target binding domain comprises a single-chain variable fragment (scFv).
15. The chimeric receptor according to any one of the preceding paragraphs, wherein the target binding domain comprises the ligand-binding domain of a nucleic acid receptor, and wherein the target is a nucleic acid.
16. A nucleic acid encoding the chimeric receptor according to any one of the preceding paragraphs.
17. A vector comprising the nucleic acid according to paragraph 16.
18. A host cell comprising the nucleic acid according to paragraph 16 or the vector according to paragraph 17.
19. A cell capable of cross-presenting an exogenous antigen, said cell comprising the host cell according to paragraph 18 and the chimeric receptor according to any one of paragraphs 1-15 expressed at the cell surface.
20. The cell according to paragraph 18 or paragraph 19, wherein the cell is a myeloid cell.
21. The cell according to paragraph 20, wherein the cell is a macrophage, a monocyte, or a dendritic cell.
22. The cell according to paragraph 18 or paragraph 19, wherein the cell is a lymphocyte.
23. The cell according to paragraph 18 or paragraph 19, wherein the cell is not a dendritic cell.
24. The cell according to paragraph 19, wherein the cell is not a professional antigen presenting cell.
25. The cell according to any one of paragraphs 19 to 24, wherein the cell is a fibroblast or a muscle cell.
26. A method of producing a cell according to any one of paragraphs 19-25, the method comprising:
   a. providing a precursor cell;
   b. introducing the nucleic acid according to paragraph 16 or the vector according to paragraph 17 into the precursor cell to produce the host cell according to paragraph 18
   c. propagating the host cell of step b. under conditions that promote expression of the chimeric receptor encoded by said nucleic acid, such that the host cell expresses the chimeric receptor and thus becomes capable of cross-presenting the exogenous antigen.
27. The method according to any one of paragraphs 19-26, wherein the exogenous antigen is the target that is bound by the target binding domain of the chimeric receptor.
28. The method according to any one of paragraphs 19-26, wherein the exogenous antigen is associated with the target that is bound by the target binding domain of the chimeric receptor.
29. A pharmaceutical composition comprising the vector according to paragraph 17 or the cell according any one of paragraphs 19-28.
30. The pharmaceutical composition according to paragraph 29, for use in a method of treating cancer, the method comprising administering the pharmaceutical composition to the patient.
31. The pharmaceutical composition according to paragraph 29, for use in a method of treating an infectious disease in a patient, the method comprising administering the pharmaceutical composition to the patient.
32. A method of treating a cancer in a patient in need thereof, the method comprising administering the pharmaceutical composition according to paragraph 29 to the patient.
33. A method of treating an infectious disease in a patient in need thereof, the method comprising administering the pharmaceutical composition according to paragraph 29 to the patient.
34. The pharmaceutical composition for use according to paragraph 30, or the method of treating a cancer according to paragraph 32, wherein the cancer is a solid tumour.
35. The pharmaceutical composition for use, or the method of treating a cancer according to paragraph 34, wherein the method comprises injecting the pharmaceutical composition into the solid tumour or into the tissue immediately surrounding the solid tumour.
36. The pharmaceutical composition according to paragraph 29, for use as a vaccine.
37. A method of vaccinating a subject, the method comprising administering the pharmaceutical composition according to paragraph 28 to a subject in need of vaccination.
38. A method of delivering a biopolymer to the cytosol of a cell, wherein the cell expresses a transmembrane protein comprising an intracellular domain that comprises a Syk-binding sequence derived from the signalling domain of the cytoplasmic tail of DNGR-1, wherein the biopolymer comprises a binding domain that can specifically bind an extracellular portion of the transmembrane protein, and wherein the method comprises contacting the cell with the biopolymer to allow the binding domain to bind to the extracellular portion of the transmembrane protein such that the biopolymer is internalised and translocated to the cytosol without being degraded in a phagosome.
39. The method according to paragraph 38, wherein the biopolymer comprises a second domain covalently joined to the binding domain.
40. The method according to paragraphs 39, wherein the binding domain is covalently joined to the second domain by a linker that can be cleaved by a protease present in the cytosol of the cell.
41. The method according to paragraph 38, wherein the biopolymer is non-covalently associated with a second biopolymer, said second biopolymer constituting a second domain.
42. The method according to any one of paragraphs 38-41, wherein the biopolymer is a protein.
43. The method according to any one of paragraphs 38-42, wherein the binding domain is an antibody.
44. The method according to any one of paragraphs 38-43, wherein the second domain is a nucleic acid, which optionally encodes an antigen.
45. The method according to paragraph 44, wherein the nucleic acid comprises a DNA that is capable of activating the cell via the STING pathway.
46. The method according to paragraph 44, wherein the nucleic acid comprises an RNA that is capable of activating the cell via the RIG-I and/or MDA5 pathways.
47. The method according to any one of paragraphs 38-42, wherein the second domain is a pro-apoptotic protein or a cytotoxin.
48. The method according to paragraph 47, wherein the pro-apoptotic protein or cytotoxin is selected from the group consisting of cytochrome C, a caspase, a maytansinoid, a dolastatin, an auristatin drug analogue, a cryptophycin, a duocarmycin deriative, an enediyne antibiotic, and pyrolobenodiazepine.
49. The method according to any one of paragraphs 38-48, wherein the transmembrane protein is a chimeric receptor according to any one of paragraphs 1-15.
50. The method according to any one of paragraphs 38-49, wherein the cell is a tumour cell.
51. The method according to any one of paragraphs 38-49, wherein the cell is an immune cell.
52. The method according to any one of paragraphs 38-51, wherein the method comprises the step of expressing the transmembrane protein in the cell before the cell is contacted with the biopolymer.
53. A method of treatment comprising the method according to any one of paragraphs 38-52.
54. The method of treatment according to paragraph 53, wherein the biopolymer comprises a tumour antigen conjugated to an anti-DNGR-1 antibody.
55. The method of treatment according to paragraph 54, wherein the biopolymer is administered to a cancer patient to elicit an anti-cancer Th1 response.
56. The method of treatment according to paragraph 55, wherein the biopolymer comprises an autoantigen and wherein the biopolymer is administered to a patient who is suffering from an autoimmune disease to elicit a tolerogenic response to the autoantigen.

### REFERENCES

A number of publications are cited above in order to more fully describe and disclose the invention and the state of the art to which the invention pertains. Full citations for some of these references are provided below. The entirety of each references mentioned anywhere in this document is expressly incorporated by reference herein, as if each were reproduced in its entirety.
1. Huang, A. Y. et al. Role of bone marrow-derived cells in presenting MHC class I-restricted tumor antigens. Science 264, 961-965 (1994).
2. Sigal, L. J., Crotty, S., Andino, R. & Rock, K. L. Cytotoxic T-cell immunity to virus-infected non-haematopoietic cells requires presentation of exogenous antigen. Nature 398, 77-80 (1999).
3. Iborra, S. et al. The DC receptor DNGR-1 mediates cross-priming of CTLs during vaccinia virus infection in mice. J Clin Invest 122, 1628-1643 (2012).
4. Zelenay, S. et al. The dendritic cell receptor DNGR-1 controls endocytic handling of necrotic cell antigens to favor cross-priming of CTLs in virus-infected mice. J Clin Invest 122, 1615-1627 (2012).
5. Smed-Sörensen, A. et al. Influenza A virus infection of human primary dendritic cells impairs their ability to cross-present antigen to CD8 T cells. PLoS Pathog 8, e1002572 (2012).
6. Alloatti, A. et al. Critical role for Sec22b-dependent antigen cross-presentation in antitumor immunity. Journal of Experimental Medicine (2017). doi:10.1084/jem.20170229
7. Theisen, D. J. et al. WDFY4 is required for cross-presentation in response to viral and tumor antigens. Science 362, 694-699 (2018).
8. Theisen, D. & Murphy, K. The role of cDC1s in vivo: CD8 T cell priming through cross-presentation. F1000Res 6, 98 (2017).
9. Huysamen, C., Willment, J. A., Dennehy, K. M. & Brown, G. D. CLEC9A is a novel activation C-type lectin-like receptor expressed on BDCA3+ dendritic cells and a subset of monocytes. J Biol Chem 283, 16693-16701 (2008).
10. Caminschi, I. et al. The dendritic cell subtype-restricted C-type lectin Clec9A is a target for vaccine enhancement. Blood 112, 3264-3273 (2008).
11. Sancho, D. et al. Tumor therapy in mice via antigen targeting to a novel, DC-restricted C-type lectin. J Clin Invest 118, 2098-2110 (2008).
12. Sancho, D. et al. Identification of a dendritic cell receptor that couples sensing of necrosis to immunity. Nature 458, 899-903 (2009).
13. Ahrens, S. et al. F-actin is an evolutionarily conserved damage-associated molecular pattern recognized by DNGR-1, a receptor for dead cells. Immunity 36, 635-645 (2012).
14. Zhang, J.-G. et al. The dendritic cell receptor Clec9A binds damaged cells via exposed actin filaments. Immunity 36, 646-657 (2012).
15. Hanč, P. et al. Structure of the Complex of F-Actin and DNGR-1, a C-Type Lectin Receptor Involved in Dendritic Cell Cross-Presentation of Dead Cell-Associated Antigens. Immunity 42, 839-849 (2015).
16. Schulz, O. et al. Myosin II Synergizes with F-Actin to Promote DNGR-1-Dependent Cross-Presentation of Dead Cell-Associated Antigens. Cell Rep 24, 419-428 (2018).
17. Fuertes Marraco, S. A. et al. Novel murine dendritic cell lines: a powerful auxiliary tool for dendritic cell research. Front Immunol3, 331 (2012).
18. Hanč, P. et al. A pH- and ionic strength-dependent conformational change in the neck region regulates DNGR-1 function in dendritic cells. EMBO J 35, 2484-2497 (2016).
19. Schnorrer, P. et al. The dominant role of CD8+ dendritic cells in cross-presentation is not dictated by antigen capture. Proc Natl Acad Sci USA 103, 10729-10734 (2006).
20. WO2009/013484A1
21. Cruz, F. M., Colbert, J. D., Merino, E., Kriegsman, B. A. & Rock, K. L. The Biology and Underlying Mechanisms of Cross-Presentation of Exogenous Antigens on MHC-I Molecules. Annu Rev Immunol (2017). doi:10.1146/annurev-immunol-041015-055254
22. Accapezzato, D. et al. Chloroquine enhances human CD8+ T cell responses against soluble antigens in vivo. J Exp Med 202, 817-828 (2005).
23. Belizaire, R. & Unanue, E. R. Targeting proteins to distinct subcellular compartments reveals unique requirements for MHC class I and II presentation. Proc Natl Acad Sci USA 106, 17463-17468 (2009).
24. Han, D. et al. Anti-tumour immunity controlled through mRNA m6A methylation and YTHDF1 in dendritic cells. Nature 566, 270-274 (2019).
25. H Zola (CRC Press, 1988) Monoclonal Antibodies: A manual of techniques
26. Böttcher, J. P. et al. Oncogenic Transformation of Dendritic Cells and Their Precursors Leads to Rapid Cancer Development in Mice. The Journal of Immunology 195, 5066-5076 (2015).
27. Thurston, T. L. M., Muhlinen, von, N. & Randow, F. Galectin 8 targets damaged vesicles for autophagy to defend cells against bacterial invasion. Nature 482, 414-418 (2012).
28. Hanč, P. et al. in (ed. Yamasaki, S.) 65-81 (Springer Japan, 2016). doi:10.1007/978-4-431-56015-9_5
29. Cebrian, I. et al. Sec22b regulates phagosomal maturation and antigen crosspresentation by dendritic cells. Cell 147, 1355-1368 (2011).
30. Lin, M. L. et al. Selective suicide of cross-presenting CD8+ dendritic cells by cytochrome c injection shows functional heterogeneity within this subset. Proc Natl Acad Sci USA 105, 3029-3034 (2008).
31. J G R Hurrell (CRC Press, 1982) Monoclonal Hybridoma Antibodies: Techniques and Applications
32. Neuberger et al (1988, 8th International Biotechnology Symposium Part 2, 792-799) discusses Chimeric antibodies
33. del Fresno, C. et al. DNGR-1 in dendritic cells limits tissue damage by dampening neutrophil recruitment. Science 362, 351-356 (2018).
34. Savina, A. et al. NOX2 controls phagosomal pH to regulate antigen processing during crosspresentation by dendritic cells. Cell 126, 205-218 (2006).
35. Graham, D. B. et al. An ITAM-signaling pathway controls cross-presentation of particulate but not soluble antigens in dendritic cells. Journal of Experimental Medicine 204, 2889-2897 (2007).
36. Savina, A. et al. The Small GTPase Rac2 Controls Phagosomal Alkalinization and Antigen Crosspresentation Selectively in CD8+ Dendritic Cells. Immunity 30, 544-555 (2009).
37. Dingjan, I. et al. Lipid peroxidation causes endosomal antigen release for cross-presentation. Sci Rep 6, 22064 (2016).
38. Gros, M. & Amigorena, S. Regulation of Antigen Export to the Cytosol During Cross-Presentation. Front Immunol 10, 1283-9 (2019).
39. Hildner, K. et al. Batf3 deficiency reveals a critical role for CD8alpha+ dendritic cells in cytotoxic T cell immunity. Science 322, 1097-1100 (2008).
40. Weimershaus, M. et al. Conventional dendritic cells require IRAP-Rab14 endosomes for efficient cross-presentation. The Journal of Immunology 188, 1840-1846 (2012).
41. Grotzke, J. E. et al. Sec61 blockade by mycolactone inhibits antigen cross-presentation independently of endosome-to-cytosol export. Proc Natl Acad Sci USA (2017).
doi:10.1073/pnas.1705242114
42. Kretzer, N. M. et al. RAB43 facilitates cross-presentation of cell-associated antigens by CD8α+ dendritic cells. Journal of Experimental Medicine 213, 2871-2883 (2016).
43. Ackerman, A. L., Giodini, A. & Cresswell, P. A role for the endoplasmic reticulum protein retrotranslocation machinery during crosspresentation by dendritic cells. Immunity 25, 607-617 (2006).
44. Zehner, M. et al. Mannose receptor polyubiquitination regulates endosomal recruitment of p97 and cytosolic antigen translocation for cross-presentation. Proc Natl Acad Sci USA 108, 9933-9938 (2011).
45. Reis e Sousa, C. & Germain, R. N. Major histocompatibility complex class I presentation of peptides derived from soluble exogenous antigen by a subset of cells engaged in phagocytosis. J Exp Med 182, 841-851 (1995).
46. Ackerman, A. L., Kyritsis, C., Tampé, R. & Cresswell, P. Early phagosomes in dendritic cells form a cellular compartment sufficient for cross presentation of exogenous antigens. Proc Natl Acad Sci USA 100, 12889-12894 (2003).
47. Guermonprez, P. et al. ER-phagosome fusion defines an MHC class I cross-presentation compartment in dendritic cells. Nature 425, 397-402 (2003).
48. Houde, M. et al. Phagosomes are competent organelles for antigen cross-presentation. Nature 425, 402-406 (2003).
49. Turk, B. & Turk, V. Lysosomes as ‘suicide bags’ in cell death: myth or reality? J Biol Chem 284, 21783-21787 (2009).
50. Zehner, M. et al. The translocon protein Sec61 mediates antigen transport from endosomes in the cytosol for cross-presentation to CD8(+) T cells. Immunity 42, 850-863 (2015).
51. Rogers, N. C. et al. Syk-dependent cytokine induction by Dectin-1 reveals a novel pattern recognition pathway for C type lectins. Immunity 22, 507-517 (2005).
52. LeibundGut-Landmann, S. et al. Syk- and CARD9-dependent coupling of innate immunity to the induction of T helper cells that produce interleukin 17. Nat Immunol 8, 630-638 (2007).
53. Nair-Gupta, P. et al. TLR signals induce phagosomal MHC-I delivery from the endosomal recycling compartment to allow cross-presentation. Cell 158, 506-521 (2014).
54. Robinson, M. J., Sancho, D., Slack, E. C., LeibundGut-Landmann, S. & Reis e Sousa, C. Myeloid C-type lectins in innate immunity. Nature Immunol. 7,1258-1265 (2006).
55. Baur and Steinle, HemITAM: A single tyrosine motif that packs a punch. Science Signaling 5 Dec. 2017: Vol. 10, Issue 508, eaan3676 DOI: 10.1126/scisignal.aan3676
56. Pan W, Clawson GA (2009) The shorter the better: reducing fixed primer regions of oligonucleotide libraries for aptamer selection. Molecules 14: 1353-1369
   For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. A chimeric receptor comprising an extracellular target binding domain, a transmembrane domain, and an intracellular domain that comprises a Syk-binding sequence derived from the signalling domain of the cytoplasmic tail of DNGR-1, wherein said Syk-binding sequence contains a tyrosine residue.

2. The chimeric receptor according to claim 1, wherein the Syk-binding sequence comprises an amino acid sequence as set forth in SEQ ID NO:15 (MHAEXXYXXLQWD) or as set forth in SEQ ID NO:90 (MHEEXXYXXLQWD).

3. The chimeric receptor according to claim 2, wherein the Syk-binding sequence comprises an amino acid sequence as set forth in SEQ ID NO:11 (MHAEEIYTSLQWD) or an amino acid sequence as set forth in SEQ ID NO:89 (MHEEEIYTSLQWD).

4. The chimeric receptor according to any one of the preceding claims, wherein the target binding domain is derived from a non-DNGR-1 lectin, a transferrin receptor, or wherein the target binding domain comprises an antibody variable region heavy chain (V_{H}) and/or light chain (V_{L}).

5. A cell comprising the chimeric receptor according to any one of claims 1-4.

6. The cell according to claim 5, wherein the cell is a professional antigen presenting cell (APC).

7. The cell according to claim 6, wherein the professional APC is a macrophage.

8. The cell according to claim 5, wherein the cell is not a professional antigen presenting cell (APC).

9. A method of delivering a biopolymer to the cytosol of a cell, wherein the cell expresses a transmembrane protein comprising an intracellular domain that comprises a Syk-binding sequence derived from the signalling domain of the cytoplasmic tail of DNGR-1, wherein the biopolymer comprises a binding domain that can specifically bind an extracellular portion of the transmembrane protein, wherein the method comprises contacting the cell with the biopolymer to allow the binding domain to bind to the extracellular portion of the transmembrane protein such that the biopolymer is internalised and translocated to the cytosol without being degraded in a phagosome, and wherein the biopolymer further comprises a nucleic acid that encodes a gene product.

10. The method according to claim 9, wherein the gene product is a pro-apoptotic protein, an enzyme, a cytotoxic peptide, or an antigen.

11. The method according to claim 10, wherein the binding domain of the biopolymer is a polypeptide that comprises an antibody variable region heavy chain (V_{H}) and/or variable region light chain (V_{L}) chain.

12. The method according to claim 9 or claim 10, wherein the second domain is covalently linked to the binding domain via a linker that can be cleaved by a protease present in the cytosol of the cell.

13. The method according to any one of claims 9 to 12, wherein the transmembrane protein is DNGR-1 and wherein the binding domain of the biopolymer specifically binds an extracellular portion of DNGR-1.

14. A biopolymer comprising a binding domain and a second domain, wherein the binding domain can specifically bind an extracellular portion of DNGR-1, and wherein the second domain is a nucleic acid that encodes a gene product.

15. The biopolymer according to claim 14, wherein the gene product is a pro-apoptotic protein, an enzyme, a cytotoxic peptide, or an antigen.

16. A nucleic acid encoding the chimeric receptor according to any one of the claim 1 to 4, or encoding the biopolymer according to claim 14 or 15.

17. A vector comprising the nucleic acid according to claim 16.

18. A cell comprising the nucleic acid according to claim 16 or the vector according to claim 17.

19. A pharmaceutical composition comprising the vector according to claim 17, or the cell according to claim 18.

20. The pharmaceutical composition according to claim 19, for use in medicine.

21. The pharmaceutical composition according to claim 19, for use for use in a method of treating cancer, the method comprising administering the pharmaceutical composition to the patient.

22. The pharmaceutical composition for the use according to claim 20, wherein the treatment elicits an anti-cancer Th1 response in the patient.

23. The pharmaceutical composition according to claim 18, for use for use in a method of treating an infectious disease, the method comprising administering the pharmaceutical composition to the infected patient.

24. The pharmaceutical composition according to claim 18, for use as a vaccine.

25. The pharmaceutical composition for the use according to any one of claims 18-22, wherein the method expresses an antigen to the cytosol of a patient cell.

26. The pharmaceutical composition for the use according to any one of claims 18-23, wherein the method activates the STING pathway, the RIG-I pathway and/or the MDA5 pathway.
